(19) United States
(12) Patent Application Publication
(10) Pub. No.: US 2023/0141670 A1
(43) Pub. Date: May 11, 2023

(54) PROCESS FOR THE SYNTHESIS OF ETHYLENE GLYCOL

(71) Applicant: FAIRBRICS SAS, Clichy (FR)

(72) Inventor: Tawfiq NASR ALLAH, Paris (FR)

(73) Assignee: FAIRBRICS SAS, Clichy (FR)

(21) Appl. No.: 17/916,221

(22) PCT Filed: Mar. 31, 2021
(86) PCT No.: PCT/IB2021/000211

§ 371 Date: Sep. 30, 2022

(30) Foreign Application Priority Data
Mar. 31, 2020 (GB) 2004665.2

(51) Int. Cl. C07C 29/149; C07C 231/10; C07C 67/36; C25B 1/23

(52) U.S. Cl. CPC C07C 29/149; C07C 231/10; C07C 67/36; C25B 1/23

(57) ABSTRACT

A process for the production of ethylene glycol from CO₂, including the steps of: i) reducing CO₂ to CO; ii) reacting the CO produced in step i) with an amine to form an oxamide or an oxamate or with an alcohol to form an oxalate; and iii) reducing the oxamide, oxamate or oxalate formed in step ii) to form ethylene glycol. Also, a process for the production of an oxamide, oxamate or oxalate and a process for the production of polyethylene terephthalate.

### FIELD OF THE INVENTION

The present invention relates to a process for the production of ethylene glycol and, in particular, to processes in which CO₂ is the feedstock. The ethylene glycol may be used in the production of polyethylene terephthalate, thereby providing a process in which CO₂ is removed from the atmosphere or removed from industrial fumes and converted into a material typically prepared from non-renewable sources.

### BACKGROUND OF THE INVENTION

Ethylene glycol (EG) is an important chemical, in particular as a monomer in the manufacture of polyethylene terephthalate (PET). PET can be formed into synthetic fibres and is the most commonly used polyester in the textiles industry, with about 49% of the world's clothing being made of polyester.

Traditionally, EG is synthesised from ethylene obtained from crude oil, such as from naptha or coal. Currently, the production of synthetic fibres, including those formed from PET, consumes 98 million tons of oil every year, which is predicted to increase to 300 million tons by 2050. However, the price of fossil fuels, and thus products made from them, are expected to increase due to both resource scarcity and increases in taxes and extracting costs. Furthermore, there is a general move towards technologies that are less reliant on non-renewable sources, and which have a lower environmental impact.

This is particularly the case in the textile industry which is one of the biggest producers of CO₂ in the world.

Research to date has predominantly focussed on recycling polyester by melting down existing plastic and re-spinning it into a new polyester fibre. This approach requires very well sorted plastics in order to produce a product with adequate mechanical properties and often requires the addition of virgin materials. It is also challenging to remove the dyes and other additives from products, making effective recycling difficult. Finally, the high temperatures typically required during the recycling process accelerates aging of the polyester, meaning that plastics can only be recycled a limited number of times and are usually only used in the production of lower quality products.

It is therefore important to develop new methods for the production EG that minimise the use of fossil fuels, allowing for greener production of PET.

The oxidative coupling of CO with amines to form oxamides or oxamates and their subsequent reduction provides an alternative synthetic route to EG. However, this has been the subject of only limited research.

The article ‘Oxidative coupling of amines and carbon monoxide catalysed by palladium complexes. Mono- and double carbonylation reactions promoted by iodine compounds’ by I. Pri-Bar and H. Alper, discloses the use of a homogeneous catalyst in combination with an iodide promoter in the synthesis of oxamides. The further use of such compounds is not discussed, nor is the origin of the CO used in the process.

WO 2010/130696 discloses the synthesis of oxamides and oxamates from CO and an amine, preferably in the presence of a catalyst, and their subsequent reduction to EG or amine analogues. However, in the examples provided, oxamides are instead synthesised from diethyl oxalate or dimethyl oxalate in the absence of a catalyst, whilst oxalic acid diamide is purchased directly. Thus, no conditions for the synthesis of oxamides or oxamates from CO and an amine are provided.

Finally, the article entitled ‘Selective catalytic two-step process for ethylene glycol from carbon monoxide’ by K Dong, S. Elangovan et al. discloses a two-step catalytic procedure involving the oxidative carbonylation of amines to oxamides and subsequent hydrogenation to EG. In the production of oxamides, a homogeneous catalyst is used in combination with a reaction pressure of approximately 5 MPa (25 bar CO and 25 bar air), with the CO described as being easily produced from natural gas, coal or biomass.

However, there are a number of drawbacks with such methods. For instance, the catalyst and product are in the same phase, which can limit the extent to which the reaction may be carried out in a continuous manner and can also make isolation of the product and reuse of the catalyst challenging. Additionally, deactivation and poisoning of the catalyst may be observed, for example due the formation of side products that poison the catalyst or because of the instability of the catalyst. For example, if phosphines are used as ligands to stabilise the catalyst, they may react with O₂ to form phosphine oxide, which in turn may decrease the efficiency of the catalyst. Finally, these reactions use CO as their starting material, which can be obtained from a variety of sources, many of which are not environmentally friendly.

Accordingly, there is a need for a process for preparing EG with lower environmental impact than the conventional methods in the art.

### SUMMARY OF THE INVENTION

The present invention uses CO₂ as the carbon source in a process for preparing EG and products derived from EG. The process may avoid the use of fossil fuel-derived reagents and thus may be more environmentally friendly and sustainable than those used in the prior art.

Accordingly, the present invention provides a process for the production of ethylene glycol from CO₂ comprising the steps of:
i) reducing CO₂ to CO;
ii) reacting the CO produced in step i) with an amine to form an oxamide or an oxamate or with an alcohol to form an oxalate; and
iii) reducing the oxamide, oxamate or oxalate formed in step ii) to form ethylene glycol.

Further provided is a process for the production of an oxamide, oxamate or oxalate, comprising the step of:
reacting CO with an amine to form an oxamide or an oxamate or with an alcohol to form an oxalate under flow conditions.

This process may be performed as described herein for step ii). This process may be combined with step i) as disclosed herein or with step iii) disclosed herein.

Thus, there is also provided a process for the production of ethylene glycol comprising the steps of:
reacting CO with an amine to form an oxamide or an oxamate or with an alcohol to form an oxalate under flow conditions; and
reducing the oxamide, oxamate or oxalate to form ethylene glycol.

The first step of this process may be performed as described herein for step ii). The second step of his process may be performed as described herein for step iii).

Also provided is a process for the production of polyethylene terephthalate comprising the steps of:
a) producing ethylene glycol according to the above process; and
b) polymerising the ethylene glycol produced in step a) with terephthalic acid or a terephthalate di-ester to produce polyethylene terephthalate.

This process may provide a greener method of producing PET which uses CO₂ as a raw material as opposed to fossil fuels.

### BRIEF DESCRIPTION OF THE DRAWINGS

The present inventions will now be described by way of example and with reference to the accompanying Figures in which:

FIG. 1 shows a ¹H NMR spectrum of EG produced according to the method described herein.

FIG. 2 shows a ¹H NMR spectrum of EG obtained from a commercial source.

FIG. 3 shows GC-MS spectra for EG produced in the present method. The mass spectrum shown is of the product present in the peak in the GC spectrum having a retention time of about 6 to 7 minutes.

FIG. 4 shows GC-MS spectra for EG obtained from a commercial source. The mass spectrum shown is of the product present in the peak in the GC spectrum having a retention time of about 6 to 7 minutes.

FIG. 5 shows a GC spectrum run in acetone of EG produced in the present method (A) and EG obtained from a commercial source (B). The EG product is present in the smaller second peak.

FIG. 6 shows an overview of an embodiment of the three-step process.

### DETAILED DESCRIPTION OF THE INVENTION

Overall Process for the Production of EG and/or PET

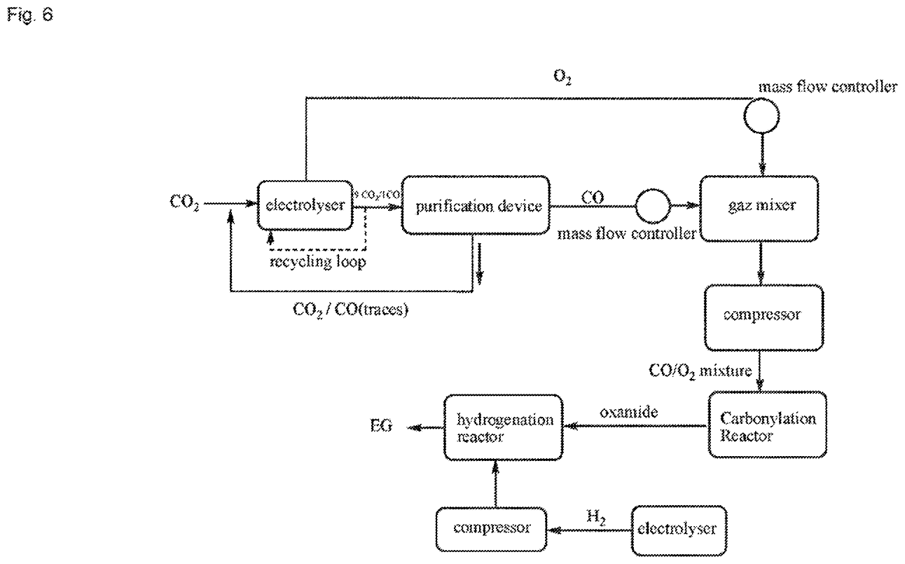

The process of the present invention provides a method of obtaining EG from CO₂ and a method for obtaining PET from EG. An overview of a three-step process according to the present invention is provided in FIG. 6.

The location where a reaction takes place is referred to as the reaction site. The reaction site may be a reaction chamber, a reaction vessel, or a tube. In embodiments, a batch reaction process is performed in a reaction chamber, autoclave or vessel. In embodiments, a flow reaction process is performed in a cartridge, tube reactor, plug flow reactor or column reactor. Preferably, a flow reaction process, in particular step ii), is performed in a packed bed reactor. The packed bed reactor preferably comprises a solid catalyst that is not soluble in the reaction mixture such as a supported homogeneous catalyst or a heterogeneous catalyst. Step i) may be performed in an electrochemical reactor. This may be designed so that it may be operated under flow conditions.

Preferably, at least two of the three steps for the production of EG may be performed in a continuous sequence in which the products from one step are introduced directly into the reaction site of the following step. The term ‘introduced directly’ is intended to mean that the process is performed without the product from one reaction being removed from and subsequently re-introduced into the apparatus in which the reactions are performed. The apparatus may comprise multiple reaction sites which are interconnected, for example by tubes. Preferably, unless otherwise described herein, the product of one step is introduced directly into the reaction site of the following step without purification of the product.

The reaction sites of the two or more steps which are performed in a continuous sequence may be the same or different reaction sites. In embodiments, the CO produced in step i) may be introduced directly into step ii), though in other embodiments it is purified and the purified CO introduced directly into step ii). In embodiments, the oxamide or oxamate produced in step ii) is introduced directly into step iii). In embodiments, all three steps for the production of EG may be performed in a continuous sequence, such that the CO produced in step i) is, optionally after purification, introduced directly into step ii) and the oxamide or oxamate produced in step ii) is introduced directly into step iii).

The reaction steps which are performed in a continuous sequence may be batch reaction processes, flow reaction processes or may include any combination of batch and flow reaction processes.

It can be beneficial if reactions are performed under continuous flow conditions (referred to as ‘flow conditions’ herein), in which the chemical reaction is run in a continuously flowing stream. The continuously flowing stream may comprise liquid and/or gas. For example, in step i), a stream preferably comprises gas, whereas in steps ii) and/or iii) a stream preferably comprises liquid. For example, when a reaction is performed under flow conditions, the reactant streams may be pumped together at a mixing junction and flowed down a temperature-controlled pipe or tube. This is in contrast to batch production, in which the chemical reaction is performed in a defined location using specified quantities of reagents which must then be transferred, often manually, to another vessel to continue the process.

Additionally, the use of flow conditions in combination with a solid catalyst, preferably in combination with a supported homogeneous or heterogeneous catalyst in step ii), avoids the need for a separate step for the separation and purification of intermediate compounds from the catalyst. Additionally, when compared to batch processes, flow processes are typically easier to understand, model and control, and scaling-up production is usually easier. Thus, the use of flow conditions is of interest in industry.

Preferably at least one, more preferably at least two and most preferably all three of the reaction steps in the process for the production of EG are performed under flow conditions. Preferably the reaction sites of steps i) ii) and iii) are connected sequentially in an order such that the product of one step is directly fed to the next step such that the reactions can be performed in a continuous sequence, as discussed above.

In an embodiment, the process of step ii) is performed under flow conditions. In embodiments, the process of steps i) and ii) may be performed under flow conditions. In another embodiment, the process of steps (ii) and (iii) may be performed under flow conditions. Preferably all of steps i), ii) and iii) are performed under flow conditions.

It has been surprisingly found that the reaction conditions of step ii) may influence the amount of solid formed during the reaction. In particular, it has surprisingly been found that under specific reactions conditions (discussed below), the amount of solid formed in step ii) is greatly reduced. It has been found that reducing solid formation in step ii) advantageously increases the yield of the reaction. In addition, solid deposition may reduce the heat transfer coefficient, reducing the temperature of the reaction solution and thus the conversion.

It is believed that, at least in part, solid formation is the result of deposition of the catalyst under the reaction conditions. This reduces the amount of catalyst available to catalyse the reaction and therefore is believed to contribute to reduced reaction yields. For example, for a homogeneous catalyst comprising palladium, it is believed that catalyst degradation to form Pd-black contributes significantly to solid formation. In addition, if phosphine oxide is present in the solid (which may result from reaction of the catalyst with oxygen as discussed hereinbelow), this may poison the catalyst used in step iii). This means that the product of step ii) would preferably be purified before performing step iii) if phosphine oxide is formed step ii), e.g. if the catalyst in step ii) comprises a phosphine.

The solid formed in this step may include salts, which can be detrimental to hydrogenation performed in step iii). These salts may, for example, be a side-product or a reagent that is not completely soluble in the reaction mixture. These may be present in large amounts and may therefore contribute to the reduced heat transfer coefficient discussed above. This means that the product of step ii) would preferably be purified before performing step iii). Thus, prevention of solid formation means that the product of step ii) can be introduced directly into step iii), preferably without purification.

Additionally, the amount of solid formed in step ii) may affect the suitability of the reaction for being performed under flow conditions. Without being bound by theory, it is believed that reducing the amount of solid produced in step ii) may prevent blockages when the reaction is performed under flow conditions. In addition, solid formation may result in pressure losses during the reaction, reducing the pumping efficiency. Reducing solid formation also prevents these problems from occurring, making the process more suitable for being performed under flow conditions.

Moreover, by reducing solid formation, the reproducibility of the process is improved, which is an important factor for scale-up of the process. In addition, less purification is required, which means that fewer process steps may be required and the cost of the process is thereby reduced.

There is also provided a process for making an oxamide, oxamate or oxalate comprising reacting CO with an amine to form an oxamide or an oxamate or with an alcohol to form an oxalate under flow conditions. This reaction may be performed under the reaction conditions described for step ii) below. As this reaction is performed under flow conditions, the same advantages described for step ii) herein are also found for this process. In particular, the conditions used in this reaction greatly affect the amount of solid formed and thus the reaction yield and its ability to be performed in flow. This process may be combined with a step of reducing the oxamide, oxamate or oxalate to form ethylene glycol. This reduction may be performed under the reaction conditions described for step iii) below.

Steps i), ii) and iii) will now be discussed individually in more detail.

Step i)

In the first step of the synthesis of EG, CO₂ is reduced to CO.

The CO₂ source may consist essentially of CO₂ or may comprise CO₂ as part of a mixture of gases. For example, the CO₂ may be provided as a mixture with an inert gas, such as nitrogen or argon. If the gas is being sourced from industrial wate, the gas may comprise H₂, CO, H₂S and/or NOₓ in addition to CO₂. Preferably, the CO₂ source consists essentially of CO₂ or comprises CO₂ as a mixture with an inert gas.

In embodiments, the CO₂ may be obtained from waste gases from industrial process. The gas comprising CO₂ collected from these processes or from alternative sources may be a gas mixture comprising one or more additional gases. This gas mixture may be purified prior to use in step i) in order to remove some or all of these additional gases. For example, the gas mixture may be purified to obtain pure CO₂. Preferably, the gas comprising CO₂ is collected from industries with major CO₂ emissions, such as large fossil fuel or biomass energy facilities, natural gas processing and/or fossil fuel-based hydrogen production plants. CO₂ may also be captured from the air. However due to the lower concentration of CO₂ in the air when compared to the abovementioned processes, such capture is more challenging and thus less preferred. By using waste CO₂ from existing processes as the CO₂ source, not only is a waste product transformed into a valuable resource, but the net CO₂ release for the combined processes may be reduced.

In embodiments, the reduction performed in step i) is electrochemical reduction. This form of reduction has been found to effectively and selectively convert CO₂ to CO. Any suitable electrodes and electrolyte may be used. This process is performed in the presence of a catalyst, such as a molecular electrocatalyst.

The anode may be a conductive electrode, preferably a carbon, platinium, glassy carbon (GC) stainless steel, silver or iron electrode. The cathode may be a carbon, mercury, glassy carbon (GC), stainless steel, silver or iron electrode.

Step (i) is preferably carried out in the presence of a proton donor, such as water, trifluoroethanol, phenol and/or acetic acid. It will be appreciated that these components will be present in the electrolyte in an electrochemical reduction. The proton donor may be present at a concentration of at least 1 mM, preferably at least 3 mM, and more preferably at least 5 mM. The proton donor may be present at a concentration of up to 120 mM, preferably up to 110 mM, and more preferably up to 100 mM. Thus, the proton donor may be present at a concentration of from 1 to 120 mM, preferably from 3 to 110 mM, and more preferably from 5 to 100 mM.

Step (i) may be carried out in the presence of water. In these instances, during the electrochemical reduction of CO₂, H₂O may be reduced at the cathode to form H₂ gas. It is generally preferred that little or no H₂ is produced in step i) so that the electrical energy used in step i) is used to produce CO. However, where H₂ gas is produced as a by-product, this H₂ gas may be recycled for use in step iii). This is particularly preferred when reduction in step iii) is catalytic hydrogenation. Preferably, the recycled H₂ is separated from CO, CO₂ and/or any other gases prior to its use in step iii). By using the H₂ produced in step i) in step iii), the energy used in converting H₂O to H₂ is not wasted, making the three step process more energy efficient.

The electrolyte further comprises one or more salts, such as n-Bu₄PF₆, TBABF₄ and/or NaCl. The electrolyte may further comprise a solvent selected from DMF, acetonitrile and/or water.

The catalyst may comprise a metal centre selected from cobalt, iron, nickel, manganese and/or rhenium. Thus the catalyst may be a molecular electrocatalyst.

The catalyst may comprise a porphyrin and/or a phthalocyanine catalyst which may be substituted or unsubstituted. The porphyrin and/or a phthalocyanine may be coordinated to a metal, preferably cobalt, iron, nickel, manganese and/or rhenium. Preferably, the porphyrin catalyst is coordinated to iron. Preferably the phthalocyanine is coordinated to cobalt.

In embodiments, the catalyst is a cobalt phthalocyanine bearing a trimenyl ammonium group appended to the phthalocyanine macrocycle (CoPc2) according to Formula I.

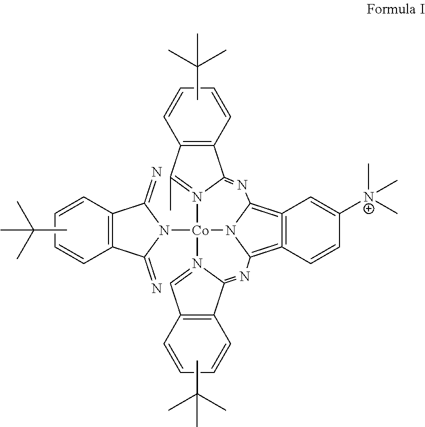

In embodiments, the catalyst is iron tetraphenylporphyrin (FeTPP), as shown in Formula II.

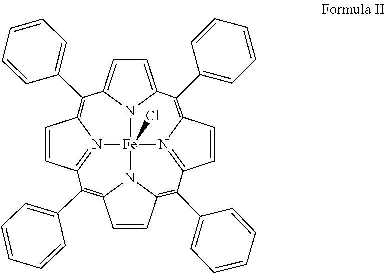

In embodiments the cathode and/or anode, preferably the cathode, comprises or consists essentially of the catalyst. For example, the electrode may comprise a coating of the catalyst.

Suitable catalysts may be any disclosed in Wang, M., et al., Nat. Commun., 10, 3602 (2019); Jensen, M. T. et al., Nat. Commun., 8, 489, (2017); Benson, E. E. et al., Chem. Soc. Rev., 38, 89-99 (2009); Costentin, C. et al., Chem. Soc. Rev., 42, 2423-2436 (2013); Costentin, C. et al., Acc. Chem. Res., 48, 2996-3006 (2015); Sampson, M. D. et al., J. Am. Chem. Soc., 138, 1386-1393 (2016); Bonin, J. et al., Coord. Chem. Rev., 334, 184-198 (2016); and Takeda, H. et al., ACS Catal., 7, 70-88 (2017).

The catalyst may be present in an amount of at least 0.0001 molar equivalents, preferably at least 0.001 molar equivalents, and more preferably at least 0.01 molar equivalents of CO₂. The catalyst may be present in an amount of up to 0.5 molar equivalents, preferably up to 0.1 molar equivalents, and more preferably up to 0.05 molar equivalents of CO₂. Thus, the catalyst may be present in an amount of from 0.0001 to 0.5 molar equivalents, preferably from 0.001 to 0.1 molar equivalents, and more preferably from 0.01 to 0.05 molar equivalents of CO₂.

The reaction may be performed under basic conditions, preferably wherein the electrolyte comprises a base, more preferably wherein the electrolyte in contact with the anode (the anolyte) comprises a base. The base may be an inorganic base, preferably an inorganic base selected from LiOH, NaOH, KOH. In embodiments, the base comprises or consists essentially of KOH.

The reaction is preferably performed at a temperature of at least 5° C., preferably at least 10° C., and more preferably at least 15° C. The reaction is preferably performed at a temperature of up to 40° C., preferably up to 35° C., and more preferably up to 30° C. Thus, the reaction is preferably performed at a temperature of from 5 to 40° C., preferably from 10 to 35° C., and more preferably from 15 to 30° C. Preferably, the reaction is performed at 25° C.

The reaction may be performed under a continuous stream of CO₂-containing gas. Preferably, the electrolyte is saturated with CO₂ during the reaction.

The potential applied to cathode during the reaction is preferably at least −2.5 V, preferably at least −1.9 V, and more preferably at least −1.3 V vs NHE. The potential applied to cathode during the reaction is preferably up to −0.5 V, preferably up to −0.7 V, and more preferably up to −0.9 V vs NHE. Thus, the potential applied to cathode during the reaction is preferably from −2.5 to −0.5 V, preferably from −1.9 to −0.7 V, and more preferably from −1.3 to −0.9 V vs NHE.

The intensity applied to the cathode during the reaction is preferably at least 1 A/m, preferably at least 1.5 A/m, and more preferably at least 2 A/m. The intensity applied to cathode during the reaction is preferably up to 6 A/m, preferably up to 5.5 A/m, and more preferably up to 5 Nm. Thus, the intensity applied to cathode during the reaction is preferably from 1 to 6 A/m, preferably from 1.5 to 5.5 A/m, and more preferably from 2 to 5 A/m.

The faradaic yield of the electrolytic reaction is preferably from 80% to 99%. This may be measured by any method known to the skilled person.

Preferably, the reaction is performed until sufficient CO is obtained to perform the reaction in step ii). In order to ensure sufficient CO is present in the gas used in step ii), the gas produced in step (i) may be purified to separate CO from other gases, as described herein. Additionally or alternatively, multiple electroreduction setups may be combined in series in order to ensure sufficient CO₂ is reduced to CO. Additionally or alternatively, the gas may be passed through the electroreduction set up in a loop in order to ensure sufficient CO₂ is reduced to CO.

The reduction of CO₂ to CO preferably results in turnover numbers (TONs) of at least 1000, preferably at least 1800, and more preferably at least 2400. The TONs may be up to 5000, preferably up to 3500, and more preferably up to 2900. Thus, the TONs may be from 1000 to 5000, preferably from 1800 to 3500, and more preferably from 2400 to 2900. TON takes its normal meaning in the art, being the number of moles of substrate that one mole of catalyst can convert before becoming inactivated.

Preferably, upon completion of the reaction, conversion of the CO₂ to CO is over 50%, preferably 70% or over, and more preferably 90% or over.

In embodiments, the CO is collected in gaseous form following reduction. This may effectively separate the gaseous produce from the other reagents, which are in solid and/or liquid form.

When collected, the gas may comprise a mixture of CO with one or more other gases. For example, the gas collected may comprise a mixture of CO and unreacted CO₂. If gases produced at the anode and the cathode are combined, for example using a gas mixer, or if a set-up is used in which the gases produced at the anode and cathode are not separated, the gas collected may comprise a mixture of CO and O₂ and/or CO₂. Following collection, the gas comprising CO may be separated from some or all other gases, e.g. from CO₂, producing pure CO or a CO and O₂ mixture, and/or may be combined with additional gases. The gases which may be combined with the gas comprising CO following collection, and optionally purification, include oxygen and inert gases, for example nitrogen and argon. For example, if CO is collected as a mixture with O₂, O₂ may be added or removed in order to achieve the desired CO to O₂ ratio. Preferably, CO is collected separately from O₂. This is possible as CO is produced at the cathode while O₂ is produced at the anode. Preferably, following collection, CO and O₂ are combined, e.g. in a gas mixer. CO and O₂ may be combined in the desired ratio for use in step ii).

Preferably CO₂ is not present in the gas used in step ii). If present in step ii), CO₂ may react with water, alcohol or amine to form carbonates, alkyl carbonates, urea and/or carbamates, and thus may generate solid side products if present in step ii). Therefore, if the gas produced in step i) comprises CO₂, it is preferably removed prior to step ii).

Purification of the CO, and optionally H₂, from other gases may be performed by any suitable method, such as using a membrane, cryogenic distillation, amine scrubbing, adsorption or via absorption. Purification of the CO may be performed in a purification module.

If H₂ is produced during step i), the gas mixture produced may comprise H₂ and CO in a molar ratio of from 1:99 to 1:1, preferably 1:95 to 50:95, more preferably 5:95 to 15:95, most preferably about 1:9. The CO:H₂ ratio may be control by the reactant flow rate, as described in ‘A perspective on practical solar to carbon monoxide production devices with economic evaluation’, Sustainable Energy Fuels, 2020, 4, 199-212.

The mixing of the gas comprising CO with additional gases may be performed in a gas mixer. The gas mixer may be connected to the reaction chamber of step i). If step i) is performed by electrochemical reduction, the reaction chamber may be an electrochemical cell and the gas mixer may be connected to the electrochemical cell. The purification module may be before the gas mixer, for example between the reaction chamber and the gas mixer. The gas mixer may further be connected to a compressor. The compressor may compress the CO gas prior to its introduction into step ii). Thus the compressor may be located between the reaction chamber of step i) and the gas mixer and/or between the gas mixer and the reaction chamber of step ii). Preferably, the compressor is located between the gas mixer and the reaction chamber of step ii).

The molar ratio of CO to O₂ in the gas exiting step i), optionally after purification, mixing of CO and O₂, and/or addition of further O₂, may be at least 1:3, preferably at least 1:1, and more preferably at least 3:1. The gas exiting step i) may comprise CO and O₂ in a molar ratio of up to 8:1, preferably 6:1, and more preferably 5:1. Thus, the gas exiting step i), preferably after exiting a gas mixer, may comprise CO and O₂ in a molar ratio of from 1:3 to 8:1, preferably from 1:1 to 6:1, and more preferably from 3:1 to 5:1. In preferred embodiments, a molar ratio of CO to O₂ of 4:1 is used as the electroreduction of CO₂ yields CO and O₂ in this ratio. The composition of the gas mixture entering step ii) is preferably controlled, for example using a gas mass flow controller.

The reduction reaction of step i) may be performed under batch or flow conditions. Preferably the reaction of step i) is performed under flow conditions.

The CO or the gas mixture comprising CO produced in step i) may be introduced directly into the reaction site of step ii), optionally after purification and/or mixing with additional gases. If a gas mixer is used in step i), the reaction site of step i) may be connected to the gas mixer, which is then connected to the reaction site of step ii). If a compressor is also present, the reaction site of step i) may be connected to the gas mixer, which is connected to a compressor that is linked to the reaction site of step ii). The product of step i) may thus be introduced directly into the reaction site of step ii).

In preferred embodiments in which step i) is an electrochemical reduction, this step is performed in an electrochemical cell. The electrochemical cell may be connected to a gas mixer. The gas mixer may optionally be connected to a compressor. The electrochemical cell, or the gas mixer or compressor if present, may be connected to the reaction site of step ii).

In embodiments in which H₂ is produced in step i), H₂ is preferably separated from other gases, as described above, and recycled for use in step iii). The H₂ produced in step i) may be introduced directly into the reaction site of step iii), optionally after purification and/or mixing with additional gases.

Step ii)

In the second step of the synthesis of EG, CO from step i) is reacted with an amine to produce an oxamide or an oxamate. A mixture of oxamide or oxamate may be produced. Preferably the product of the reaction is an oxamide.

An oxamate may be obtained if CO is reacted with an amine in the presence water or an alcohol, e.g. if water or an alcohol is present as a solvent.

The oxamide or oxamate of the present invention preferably has the structure shown in Formula IIIa:

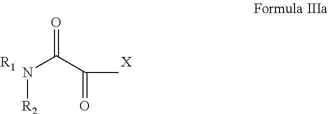

wherein X is either OR₃ or NR₃R₄ and R₁, R₂, R₃ and R₄ may be independently selected from H, an alkyl and/or an aryl group. R₁ and R₂ may be connected to each other such that NR₁R₂ forms a cyclic alkyl or aryl group including the nitrogen atom. In embodiments in which X is NR₃R₄, R₃ and R₄ may be connected to each other such that NR₃R₄ forms a cyclic alkyl or aryl group including the nitrogen atom. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. In embodiments, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted. R₁, R₂, R₃ and/or R₄ may be benzyl groups.

Preferably, X is NR₃R₄ and R₁ and R₂ are connected to each other such that NR₁R₂ form an aliphatic or aromatic amine, preferably selected from aniline, piperidine, morpholine or pyrrolidine, and R₃ and R₄ are similarly connected to each other such that NR₃R₄ form an aliphatic or aromatic amine, preferably selected from aniline, piperidine, morpholine or pyrrolidine.

In embodiments in which X is NR₃R₄, the product is an oxamide. In embodiments in which X is OR₃, the product is an oxamate.

Alternatively, CO from step i) is reacted with an alcohol to produce an oxalate.

The oxalate of the present invention preferably has the structure shown in Formula IIIb:

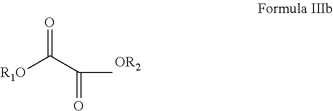

wherein R₁ and R₂ may be independently selected from H, an alkyl and/or an aryl group. The oxalate is preferably a dialkyl oxalate, i.e. R₁ and R₂ are alkyl groups. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. In embodiments, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted. Typically R₁ and R₂ will be the same, unless a mixture of different alcohols is used to prepare the oxalate.

Though generally less preferred, CO may be reacted with a mixture of an amine and an alcohol to produce a mixture of oxamide, oxamate and/or oxalate, such as a mixture of oxamide and oxalate, oxamate and oxalate, or oxamide, oxamate and oxalate.

This reaction is performed in the presence of a catalyst. The catalyst may be a homogenous, heterogeneous or supported homogeneous catalyst. In preferred embodiments, the catalyst is a supported homogeneous catalyst. In other preferred embodiments the catalyst is a heterogeneous catalyst.

Previous syntheses of oxamides, oxamates and/or oxalates use homogeneous catalysts, which often demonstrate high activities and/or selectivities. However, these catalysts may be difficult to remove from the reaction mixture and are often not recovered intact. Thus, although high activities and/or selectivities may be achieved, such catalysts and conditions may not be applicable on an industrial scale.

Preferably, the catalyst is a heterogeneous catalyst. Heterogeneous catalysts allow for easy separation from the reaction mixture, allowing for their recovery and re-use. Heterogeneous catalysts have also been found to be easier to synthetise and purify. In addition, heterogeneous catalysts have been found to be more stable and cheaper than homogeneous catalysts.

Supported homogeneous catalysts provide many benefits over homogeneous and heterogeneous catalysts as they allow for easy separation from the reaction mixture, analogous to heterogeneous catalysts, whilst retaining the high activity and selectivity usually associated with homogeneous catalysts. However, such a catalyst has not previously been investigated for the reaction of CO with an amine to produce an oxamide or an oxamate as it can be difficult to synthesise effective supported homogeneous catalysts. By attaching the homogeneous catalyst to a support, the reactivity of the catalyst can be affected as the support interferes (e.g. electronically and/or sterically) with the reaction. Additionally, supporting the homogeneous catalyst often requires modification of the catalyst itself in order to allow it to be bound to the support. For example, as discussed further below, if the homogeneous catalyst is a metal complex, one of the ligands may be modified such that it can be bound to the support in addition to being coordinated to the metal centre. The effect of such modification is not always predictable.

It has now been found that, despite these difficulties, supported homogeneous catalysts can be used to successfully catalyse this reaction.

A supported homogeneous catalyst is known in the art to comprise a homogeneous catalyst which is retained in a solid state by being bonded to, encapsulated within, or in some way associated with a solid support. This means that the homogeneous catalyst is retained in a solid state during and after the reaction, making it readily isolatable from the reaction mixture.

In embodiments, the homogeneous catalysts of the present invention comprise metal complexes which are composed of a metal centre coordinated to one or more ligands. In embodiments, the metal centre is a Group X metal. Preferably the metal centre is palladium.

These metal complexes may be associated with a solid support in various ways. In one embodiment, the metal complex is bound to the support via one or more of its ligands. This method provides the advantage that the catalytic metal centre is easily accessible during the reaction.

The ligand or ligands which are both bound to the support and coordinated to the metal centre preferably comprise a first end which is coordinated to the metal centre and a second end which is bound to the solid support. In embodiments, the first end of the ligand does not bond to the solid support, whilst the second end of the ligand does not coordinate to the metal centre. The first and second ends of the ligand may be the same functional group as one another or different functional groups. The term ‘functional group’ takes its normal meaning as a substituent or moiety within a molecule and may comprise one or more atoms. Where more than one ligand is present, the ligands may be identical or different.

The ligand may be bound to the solid support via any means of chemical bonding, including covalent bonds, ionic bonds and/or intermolecular interactions such as hydrogen bonding. Preferably the metal complex is covalently or ionically bound to the solid support, with covalent bonding being the most preferred. In embodiments, the ligand may be covalently bound to the support by a carbon atom or a nitrogen atom.

The ligand may be coordinated to the metal centre through a functional group comprising nitrogen, oxygen, phosphorous and/or a carbon. In embodiments, the ligand is coordinated to the metal centre through one or more of these atoms.

In embodiments, the ligand comprises a phosphorous atom, which is coordinated to the metal centre. Preferably, the ligand comprises a phosphine functional group which is coordinated to the metal centre through the phosphorous atom.

The ligand may have any denticity when it is coordinated to the metal centre, for example it may be a monodentate, bidentate or tridentate ligand.

In embodiments, the ligand or ligands which are both bound to the support and coordinated to the metal centre may have the structure for Formula IV:

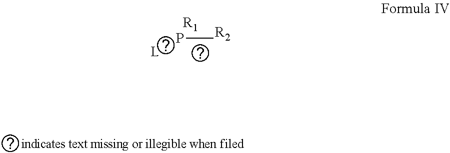

wherein R₁ and R₂ may be independently selected from H, an alkyl and/or an aryl group. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. In embodiments, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted. Preferably R₁ and R₂ are aryl groups, more preferably R₁ and R₂ are phenyl groups; and wherein L is a group linking the phosphorous atom to the solid support. L may include an alkyl and/or an aryl group. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. In embodiments, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted. In embodiments, L may comprise a nitrogen or a carbon atom, for example L may be —CH₂, —(CH₂)₂—, —(CH₂)ₙ—, —CHR₃, —CR₃R₄, —(CH)—OR₃, —(CH)R₃, —(CH)OH, —(CH)(CH₂)ₙOH, —(CH)NR₃R₄, —(CH)(CH₂)ₙNR₃R₄ and/or —(CH)(CH₂)ₙSi(OR₃)₃ wherein R₃and R₄ are as defined above for R₁ and R₂ and n may be from 1 to 10, preferably 1 to 5.

In embodiments, the ligand or ligands which are both bound to the support and coordinated to the metal centre may have the structure of Formula V:

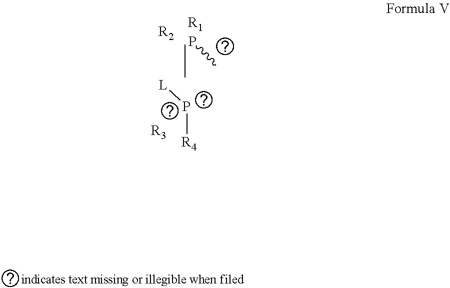

wherein R₁, R₂, R₃ and R₄ may be independently selected from H, an alkyl and/or an aryl group. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. In embodiments, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted. Preferably R₁, R₂, R₃ and R₄ are aryl groups, more preferably R₁, R₂, R₃ and R₄ are phenyl groups. In embodiments PR₁R₂ and PR₃R₄ are the identical; and wherein L is a group linking the phosphorous atom to the solid support. L may include an alkyl and/or an aryl group. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. In embodiments, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted. In embodiments, L may comprise a nitrogen or a carbon atom, for example L may be —CH₂, —(CH₂)₂—, —(CH₂)ₙ—, —CHR₅, —CR₅R₆, —(CH)—OR₅, —(CH)R₅, —(CH)OH, —(CH)(CH₂)ₙOH, —(CH)NR₅R₆, —(CH)(CH₂)ₙNR₅R₆ and/or —(CH)(CH₂)ₙSi(OR₅)₃ wherein R₅ and R₆ are as defined above for R₁, R₂, R₃ and R₄ and n may be from 1 to 10, preferably 1 to 5.

In both Formula IV and V, the phosphorous atom is coordinated to the metal centre along the wavy line and the L group is bound to the solid support (bond not shown).

In embodiments, the ligand does not comprise phosphorous. Preferably the catalyst does not comprise phosphine ligands. Phosphorous may oxidise in the presence of oxygen. This reaction may result in the loss of catalyst activity. Therefore, it may be beneficial to avoid the use of phosphorous-containing catalysts in order to prevent side-reactions. In particular, the oxidation of phosphines may lead to the formation of phosphine oxide by-products. If phosphine oxide is present in the product of step ii), the yield of hydrogenation in step iii) may be reduced if the phosphine oxide is not removed prior to step iii). Therefore, phosphine oxide is preferably removed prior to step iii).

The metal centre may also be coordinated to one or more additional ligands which are not bound to the solid support. In embodiments, one or more of these additional ligands are selected from acetate, a halogen, a solvent molecule, CO or an N-heterocyclic carbene (NHC).

In embodiments in which the metal centre is palladium, the catalyst may comprise a ligand of Formula IV and three additional ligands, or a ligand of Formula V and two additional ligands. These additional ligands are preferably acetate.

Preferably, the ligand comprises a N-heterocyclic carbene (NHC), which is coordinated to the metal centre. Preferably this ligand type is used when the ligands do not comprise phosphines, more preferably the ligand does not comprise phosphorous. NHCs are less sensitive to oxygen than phosphines, therefore do not react with the oxygen present in step ii). This means that catalyst activity is retained and/or that the amount of solid residue is reduced when compared to catalysts comprising phosphine ligands.

The ligand may have any denticity when it is coordinated to the metal centre, for example it may be a monodentate, bidentate or tridentate ligand.

In embodiments, the ligand or ligands which are both bound to the support and coordinated to the metal centre may have the structure for Formula VI:

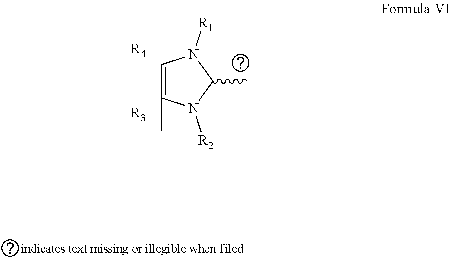

wherein R₁, R₂, R₃ and R₄ may be independently selected from H, an alkyl and/or an aryl group. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. In embodiments, alkyl groups are unsubstituted and do not comprise heteroatoms. In alternative embodiments, alkyl groups are unsubstituted and comprise one or more heteroatoms. Suitable aryl groups include five-membered to twelve-membered aromatic rings, which may comprise one or more heteroatoms, for example nitrogen and/or oxygen. Aryl groups may be substituted or unsubstituted. Preferably R₁, R₂, R₃ and R₄ are alkyl groups.

In embodiments, one or more of R₁, R₂, R₃ or R₄ may be a group linking the ligand to the solid support. R₁, R₂, R₃ and R₄ may comprise a nitrogen or/and oxygen atom, for example R₁, R₂, R₃ and Ra may be independently selected from —(CH₂)—OR₅, —(CH₂)ₙR₅, —CHR₅R₆, —(CH₂)ₙOH, —(CH₂)ₙNR₅R₆, —(CH₂)ₙSR₅R₆, and/or —(CH₂)ₙSi(OR₅)₃, wherein R₅ and R₆ may be independently selected from H, an alkyl and/or an aryl group and n may be from 1 to 10, preferably 1 to 5. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. In embodiments, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted.

In embodiments, the ligand or ligands which are both bound to the support and coordinated to the metal centre may have the structure of Formula VII:

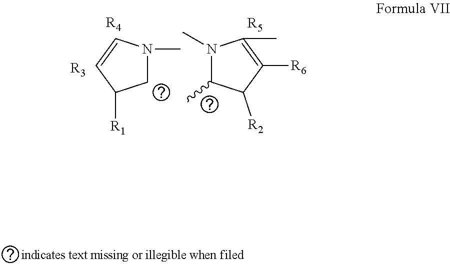

wherein R₁, R₂, R₃, R₄, R₅ and R₆ may be independently selected from H, an alkyl and/or an aryl group. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. In embodiments, alkyl groups are unsubstituted and do not comprise heteroatoms. In alternative embodiments, alkyl groups are unsubstituted and comprise one or more heteroatoms. Suitable aryl groups include five-membered to twelve-membered aromatic rings, which may comprise one or more heteroatoms, for example nitrogen and/or oxygen. Aryl groups may be substituted or unsubstituted. Preferably R₁, R₂, R₃ and R₄ are alkyl groups. In embodiments R₁, R₃ and R₄ are identical and R₂, R₅ and R₆ are identical; and wherein one or more R group is linking the carbon atoms and/or the nitrogen atom to the solid support.

In embodiments, one or more of R₁, R₂, R₃, R₄, R₅ and R₆ may be a group linking the ligand to the solid support. R₁, R₂, R₃, R₄, R₅ and R₆ may comprise a nitrogen or/and oxygen atom, for example R₁, R₂, R₃, R₄, R₅ and R₆ may be independently selected from —(CH₂)—OR₇, —(CH₂)ₙR₇, —CHR₇R₈, —(CH₂)ₙOH, —(CH₂)ₙNR₇R₈, —(CH₂)ₙSR₇ and/or —(CH₂)ₙSi(OR₇)₃, wherein R₇ and R₈ may be independently selected from H, an alkyl and/or an aryl group and n may be from 1 to 10, preferably 1 to 5. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. In embodiments, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted.

In both Formula VI and VII, the carbon atom is coordinated to the metal centre along the wavy line and one or more of the R group is bound to the solid support (bond not shown).

The metal centre may also be coordinated to one or more additional ligands which are not bound to the solid support. In embodiments, one or more of these additional ligands are selected from acetate, a halogen, a solvent molecule, CO, amine, phosphine, alcohol, or an N-heterocyclic carbene (NHC).

In an alternative embodiment, the homogeneous catalyst may be encapsulated in the voids of a porous support to form a supported homogeneous catalyst. This may be achieved by impregnation of the solid support. This method of supporting the homogeneous catalyst provides the advantage of preventing the catalytic species from dimerising or aggregating under reaction conditions.

Preferably, the metal complex is bound to the support via one or more of its ligands.

In embodiments, the solid support of the supported homogeneous catalyst does not catalyse the reaction between CO and an amine to form an oxamide and an oxamate. The support is preferably inert under the reaction conditions and does not interfere with the reaction. Preferably the support has no pronounced surface acidity, which may induce secondary reactions. In embodiments, the support provides surface groups which allows for bonding of the second end of the ligand of the metal complex.

The solid support may be any suitable solid support and is not particularly limited. The support may comprise an inorganic oxide, a polymer or carbon. In embodiments, the support comprises silica, alumina, a transition metal oxide, a polymer, carbon or mixtures thereof. Transition metal oxides include titania and zirconia. Preferably, the support is selected from a polymer or silica. Preferably the support neither shrinks, swells nor dissolves in the reaction solvent, and preferably in any solvent. The polymer can be tailored to achieve the desired properties.

Suitable catalysts include polymer-bound polymer-bound FibreCat® di(acetato)dicyclohexylphenylphosphinepalladium(II) (available from Sigma Aldrich), polymer-bound dichlorobis(triphenylphosphine)palladium(II) (available from Sigma Aldrich), polymer-bound bis[(diphenylphosphanyl)methyl]amine palladium(II) acetate (available from Sigma Aldrich) and/or siliaCat DPP-Pd Heterogeneous Catalyst (R390-100) (available from SiliCycle).

In embodiments, the catalyst may be a homogeneous catalyst. As defined above, the homogeneous catalysts of the present invention may comprise metal complexes which are composed of a metal centre coordinated to one or more ligands. In embodiments, the metal centre is a Group VIII, IX or X metal, preferably a Group VII or X metal. Preferably, the metal is selected from palladium, nickel, iron, cobalt, rhodium or iridium, more preferably palladium, nickel or iron.

The one or more ligands may be any suitable ligand. The ligands may be as defined above for supported homogeneous catalysts without being bound to a solid support. Thus, the ligands may be as defined above, but without the group for linking the ligand to the solid support. For example, a ligand may have Formula IV or V, wherein L is not linked to a solid support. Thus, L need not include the functional group for linking the ligand to a solid support. Alternatively, a ligand may have Formula VI or VII wherein the R groups are not linked to a solid support. Thus, the R groups need not include the functional group for linking the ligand to a solid support.

Preferably, the metal centre is coordinated to one or more ligands selected from acetate, a halogen, a solvent molecule, CO or an N-heterocyclic carbene (NHC), preferably acetate and/or CO. The catalyst comprises the combination of any of these ligands, preferably the combination of CO and NHC. Suitable catalysts may comprise the combination of one or more of these ligands with a phosphine.

Preferably, the catalyst comprises a carbonyl complex of the metals listed above, for example a palladium, nickel or iron carbonyl complex. Suitable catalysts include Pd(acac), Ni(CO)₄, Fe₃(CO)₁₂, Fe(CO)₅, or combinations thereof.

In embodiments, the catalyst may be a heterogeneous catalyst. Preferably, the heterogeneous catalyst comprises a metal, preferably the metal is a Group VIII to XI metal, more preferably a metal selected from Ag, Fe, Co, Ni, Ru or Pd. It has been surprisingly found that heterogeneous catalysts comprising Ag, Fe, Co, Ni and Ru effectively catalyse the formation of oxamides, oxamates or oxalates from the reaction of CO and an amine or an alcohol. To the best of our knowledge, these metals are not known to catalyse this reaction, in particular not when used in catalytic amounts such as the amounts described herein.

The metal is preferably supported on a solid support. The solid support may be any suitable solid support and is not particularly limited. The support may comprise an inorganic oxide, a polymer or carbon. In embodiments, the support comprises silica, alumina, a transition metal oxide, a polymer, carbon or mixtures thereof. Transition metal oxides include titania and zirconia. Preferably, the support is selected from silica, alumina or carbon. Alumina or silica may be calcinated. Preferably the support neither shrinks, swells nor dissolves in the reaction solvent, and preferably in any solvent. The polymer can be tailored to achieve the desired properties.

The metal is preferably present in the heterogeneous catalyst in an amount of at least 1 wt %, preferably at least 2 wt. %, more preferably at least 4 wt % of the mass of the catalyst. The metal is preferably present in the heterogeneous catalyst in an amount of up to 10 wt %, preferably at least 8 wt. %, more preferably at least 6 wt % of the mass of the catalyst. Thus, the metal may be present in the heterogeneous catalyst in an amount of from 1 to 10 wt %, preferably from 2 to 8 wt. %, more preferably from 4 to 6 wt % of the mass of the catalyst.

Suitable catalysts include Pd/Al₂O₃, Pd/C, Ag/Al₂O₃, Co/SiO₂, Co/Al₂O₃, Fe/Al₂O₃ and/or Ni/Al₂O₃.

The catalyst may be present in an amount of at least 0.00001 (1×10⁻⁵) molar equivalents, preferably at least 0.00005 (5×10⁻⁵) molar equivalents, and more preferably at least 0.0001 (1×10⁻⁴) molar equivalents of the amine or alcohol. The catalyst may be present in an amount of up to 0.1 molar equivalents, preferably up to 0.075 molar equivalents, and more preferably up to 0.05 molar equivalents of the amine or alcohol. Thus, the catalyst may be present in an amount of from 0.00001 to 0.1 molar equivalents, preferably from 0.00005 to 0.075 molar equivalents, and more preferably from 0.0001 to 0.05 molar equivalents of the amine or alcohol.

For completeness, it is noted that for heterogeneous and supported homogeneous catalysts, the catalyst amounts are defined with reference to the catalytic metal and/or complex only and does not include the mass of the support.

The reaction is performed under an atmosphere comprising CO. The atmosphere may comprise an oxidant, which may be selected from O₂, an iodide derivative such as I₂, a metal or metal complex such as CuCl₂, 1,4-dichlorobenzene and/or 1,4-benzoquinone. Preferably, the oxidant comprises O₂. The atmosphere may comprise an inert gas, such as N₂ or Ar. In embodiments, the atmosphere comprises CO without an oxidant. In alternative embodiments, the atmosphere comprises CO with an oxidant. Preferably the atmosphere consists essentially of CO or a mixture of CO and O₂.

The atmosphere may comprise CO and O₂ in a molar ratio of at least 1:3, preferably at least 1:1, more preferably at least 3:1, and most preferably at least 3.5:1. The atmosphere may comprise CO and O₂ in a molar ratio of up to 6:1, preferably 6.5:1, more preferably 5:1, and most preferably 4.5:1. Thus, the atmosphere may comprise CO and O₂ in a molar ratio of from 1:3 to 6:1, preferably from 1:1 to 6.5:1, more preferably from 3:1 to 5:1, and most preferably from 3.5:1 to 4.5:1. Preferably the molar ratio of CO to O₂ is about 4:1.

The reaction of step ii) is preferably performed under an atmosphere comprising a molar excess of CO compared to the amine or alcohol. The molar ratio of CO in the atmosphere to amine or alcohol may be at least 2:1, preferably at least 3:1, and more preferably at least 4:1. The molar ratio of CO in the atmosphere to amine or alcohol may be up to 12:1, preferably 10:1, and more preferably 9:1. Thus, the molar ratio of CO in the atmosphere to amine or alcohol may be from 2:1 to 12:1, preferably from 3:1 to 10:1, and more preferably from 4:1 to 9:1.

The reaction is preferably performed at a pressure of at least 0.1 MPa, preferably at least 0.5 MPa, more preferably at least 1 MPa, and most preferably 2 MPa. The reaction is preferably performed at a pressure of up to 10 MPa, preferably up to 8 MPa, more preferably up to 6 MPa, and most preferably up to 5 MPa. Thus, the reaction is preferably performed at a pressure of from 0.1 to 10 MPa, preferably from 0.5 to 8 MPa, more preferably from 1 to 6 MPa, and most preferably from 2 to 5 MPa.

The reaction is preferably performed at a temperature of at least 25° C., preferably at least 30° C., and more preferably at least 40° C. The reaction is preferably performed at a temperature of up to 150° C., preferably up to 110° C., and more preferably up to 80° C. Thus, the reaction is preferably performed at a temperature of from 25 to 150° C., preferably from 30 to 110° C., and more preferably from 40 to 100° C. Preferably, the reaction is performed at a temperature of from 25 to 100° C.

When the reaction is performed under batch conditions, the reaction may be performed for at least 0.1 hours, preferably at least 1 hour, more preferably at least 6 hours, and most preferably at least 16 hours. The reaction may be performed for up to 72 hours, preferably up to 48 hours, more preferably up to 30 hours, and most preferably up to 25 hours. Thus, the reaction may be performed for a duration of from 0.1 to 72 hours, preferably from 1 to 48 hours, more preferably from 6 to 30 hours and most preferably from 16 to 25 hours. The reaction may be considered complete once this time period has elapsed.

When the reaction is performed under flow conditions, the residence time is at least 30 seconds, preferably at least 5 minutes, more preferably at least 10 minutes such as at least 15 minutes. The residence time may be up to 60 minutes, preferably up to 45 minutes, more preferably up to 35 minutes. Thus, the residence time may be performed for a duration of from 30 seconds to 60 minutes, preferably from 5 to 25 minutes, more preferably from 10 to 35 minutes, for example 15 to 35 minutes.

Preferably, upon completion of the reaction, conversion of the amine or alcohol is over 50%, preferably 70% or over, and more preferably 90% or over.

The reaction may be passed through a series of reactors. Additionally or alternatively, the reaction mixture may be passed through the reactor in a loop. Thus, the system can achieve the desired conversions, preferably complete conversion, if this is not achieved initially.

The reaction of step ii) may be carried out in the presence of a solvent system. The solvent system may comprise THF, toluene, acetonitrile, DMF, dioxane, NMP, methanol, ethanol, and mixtures thereof. It will be appreciated that a wide range of other solvents may be used in step (ii). In embodiments, the solvent system comprises THF, toluene, acetonitrile and/or dioxane. In preferred embodiments the solvent system consists essentially of THF or toluene. The amount of the solvent system used is not particularly limited. In embodiments, from 50 to 6000 mL, preferably from 75 to 1000 mL, and more preferably from 100 to 900 mL of the solvent system may be used. Preferably this amount of solvent is used per mole of amine or alcohol as this ensures that the concentration of the reagents and catalysts are such that solid formation is reduced in step ii).

The amine used in the production of the oxamide or oxamate is not particularly limited provided it can react with CO. Preferably, the amine is a secondary amine as these have been found to result in lower quantities of urea by-product. The amine may have the formula NHR₁R₂, wherein R₁ and R₂ may be independently selected from H, an alkyl and/or an aryl group. R₁ and R₂ may be connected to each other such that NR₁R₂ forms a cyclic akyl or aryl group including the nitrogen atom. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. Preferably, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted. R₁, R₂, R₃ and/or R₄ may be benzyl groups. Preferably, R₁ and R₂ are independently selected from H, an alkyl group or a benzyl group, preferably a C₁ to C₆ branched or linear alkyl group or a benzyl group. In embodiments, the amine is a cyclic amine, preferably a cyclic non-aromatic amine, more preferably an amine selected from piperidine, morpholine, pyrrolidine, piperazine and derivatives thereof.

The alcohol used in the production of the oxalate is not particularly limited provided it can react with CO. The alcohol may have the formula R₁OH, wherein R₁ is selected from H, an alkyl and/or an aryl group. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. Preferably, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted. Preferably, R₁ is selected from H or an alkyl group, preferably a C₁ to C₆ branched or linear alkyl group. In embodiments, the alcohol is selected from methanol, ethanol, propanol, isopropanol, butanol, and derivatives thereof, preferably ethanol. It is generally preferred that a single alcohol will be used to produce the oxalate and so the R groups in the oxalate will be the same, e.g. if ethanol is used the oxalate will be diethyl oxalate.

Unless otherwise stated, the quantities of other reagents are measured against the quantity of the amine or alcohol. Thus, the amine or alcohol will be present in 1 molar equivalent.

The reaction may be performed in the presence of a base. Without being bound by theory, the base is believed to play a role in the equilibrium between the amine and the ammonium salt. However, it has been found that the presence of base is not needed for the reaction to be performed in good yields. It has also been found that the presence of base can lead to solid formation, for example due to the low solubility of the base in the reaction mixture. Solid formation may be detrimental to the reaction yield and may cause problems with the reaction process such as causing blockages, reducing heat transfer and reducing the pressure of the system, as discussed in detail above. This is particularly detrimental when the reaction is performed under flow conditions. The use of base also means that the base may need to be removed prior to the next stage. Preferably base is not used as this makes purification simpler and cheaper and/or addresses the problems associated with solid formation.

The identity of the base is not limited provided it does not otherwise interfere with the reaction. In embodiments, the base may be pyridine, Ca(OH)₂, K₂CO₃, LiOH, NaOH, KOH, ^{t}BuOLi, ^{t}BuONa and/or ^{t}BuOK, preferably NaOH, KOH and/or ^{t}BuOK. Preferably, bases that are solid at 25° C., in particular K₂CO₃, are not used as the base as these may lead to solid formation.

The base may be present in an amount of at least 0.01 molar equivalents, preferably at least 0.02 molar equivalents, and more preferably at least 0.03 molar equivalents of the amine or alcohol. The base may be present in an amount of up to 0.4 molar equivalents, preferably up to 0.1 molar equivalents, and more preferably up to 0.6 molar equivalents of the amine or alcohol. Thus, the base may be present in an amount of from 0.01 to 0.4 molar equivalents, preferably from 0.02 to 0.1 molar equivalents, and more preferably from 0.03 to 0.06 molar equivalents of the amine or alcohol.

More preferably, the reaction is performed in the absence of base. It has been found that when a base is present, in particular K₂CO₃, a large part of the base may not dissolve in the reaction mixture. This means that solid salt is present in the reaction mixture. Thus, at the end of the reaction, a pre-purification is preferably performed to eliminate these solids from the reaction mixture.

If the base is sparing soluble in the reaction mixture at low temperatures, for example at 25° C., the reaction mixture may be heated to a temperature at which the base is soluble in the reaction mixture. The reaction is preferably then performed at this temperature and optionally the product further processed at this temperature in order to ensure the base remains soluble in the reaction mixture and solid is not deposited. This may also apply to other additives, such as promotors, that are sparingly soluble in the reaction mixture at low temperature but which are soluble at elevated temperatures.

The reaction may be performed in the presence of one or more promotors.

The promotors may comprise iodine, an iodide derivative, an ammonium salt, or combinations thereof. In embodiments, the iodide derivative may comprise I₂, KI, LiI, HI, NaI, ⁿBu₄NI or an ammonium salt which may have the formula [NR₁R₂R₃R₄]ₙX, wherein R₁, R₂, R₃ and R₄ and may be independently selected from H, an alkyl and/or an aryl group. Suitable alkyl groups include linear or branched, cyclic or non-cyclic C₁ to C₁₀ chains. Alkyl groups may be substituted or unsubstituted and may comprise one or more heteroatoms, for example nitrogen and/or oxygen. Preferably, alkyl groups are unsubstituted and do not comprise heteroatoms. Suitable aryl groups include five-membered to twelve-membered aromatic rings, which may comprise one or more heteroatoms, for example nitrogen and/or oxygen. Aryl groups may be substituted or unsubstituted. Preferably, R₁, R₂, R₃ and R₄ are independently selected from H or an alkyl group, preferably a C₁ to C₆ branched or linear alkyl group. X may be any negatively charged ion, preferably a halide ion, more preferably iodide. ⁿBu₄NI has been found to have good solubility at 25° C., and thus does not precipitate during the reaction. Thus, the use of ⁿBu₄NI as promotor may result in less solid formation. Preferably, the promotor is ⁿBu₄NI. Other iodide salts such as KI, LiI and NaI also have good solubility and thus may be used in the present invention without resulting in excessive solid formation, however they are not as soluble as ⁿBu₄NI.

In embodiments, the promotor may be present in an amount of at least 0.005 molar equivalents, preferably at least 0.01 molar equivalents, and more preferably at least 0.02 molar equivalents of the amine or alcohol. The promotor may be present in an amount of up to 0.075 molar equivalent, preferably up to 0.05 molar equivalents, and more preferably up to 0.03 molar equivalents of the amine or alcohol. Thus, the promotor may be present in an amount of from 0.005 to 0.075 molar equivalents, preferably from 0.01 to 0.05 molar equivalents, and more preferably from 0.02 to 0.03 molar equivalents of the amine or alcohol. Preferably, the promotor is present in an amount of 0.025 molar equivalents to the amine or alcohol.

It has been found that performing step ii) under the above conditions prevents solid formation during the reaction process. Preferably, step ii) is performed using one or more conditions selected from 0.00001 to 0.05 molar equivalents of catalyst with respect to the amine or alcohol, CO and O₂ in a molar ratio of from 3.5:1 to 4.5:1, a pressure of from 2 to 6 MPa, a temperature of from 25 to 100° C., 100 to 900 mL of the solvent system per mole of amine or alcohol, and a promotor in an amount of from 0.02 to 0.03 molar equivalents of the amine or alcohol. In addition, if the process is a batch reaction, the duration is preferably from 6 to 25 hours, and if the reaction is a flow process the residence time is preferably from 15 to 35 minutes. Preferably the reaction is performed under flow conditions and the residence time is from 15 to 35 minutes. Preferably, the reaction is performed using all of these conditions in order to ensure solid is not formed.

In embodiments, K₂CO₃ is not present in the reaction, preferably no base is present, and the promotor is used in an amount of 0.02 to 0.03 molar equivalents of the amine or alcohol, preferably 0.025 molar equivalents. These conditions have been found to be particularly effective at preventing solid formation, in particular preventing base and/or salt precipitation. Additionally or alternatively, CO and O₂ are used in a molar ratio of from 3.5:1 to 4.5:1, a pressure of from 2 to 5 MPa and a temperature of from 25 to 100° C. is used as this prevents precipitation of solid catalyst particles, in particular solid metal particles, for example Pd(0) particles when the catalyst comprises palladium. The use of the combination of all of these conditions avoids catalyst precipitation, the formation of insoluble salts or the formation of side products.

If the catalyst does not comprise phosphines, e.g. phosphine ligands are not used if the catalyst is a homogeneous or supported homogeneous catalyst, base is not used, e.g. K₂CO₃ is not present in the reaction, and a promotor is used in an amount of up to 0.03 molar equivalents of the amine or alcohol, e.g. from 0.02 to 0.03 molar equivalents of the amine or alcohol, preferably wherein the promotor is ⁿBu₄NI, hydrogenation in step iii) may be performed without further purification of the reaction mixture. This means that fewer process steps are required, making the process simpler and/or cheaper. If phosphine oxide is present in the product of step ii), the yield of hydrogenation in step iii) may be reduced if the phosphine oxide is not removed prior to step iii). This may also reduce the yield of the reaction in step ii). Therefore, if phosphines are present in the catalyst of step ii), phosphine oxide may be produced which is preferably removed prior to step iii). More preferably, the solvent is THF and the solvent is not changed prior to performing step iii). This again makes the process simpler. This also facilitates performing the reaction under flow conditions as the solvent need not be replaced between steps ii) and iii).

Preventing solid formation is advantageous as it increases the yield of the reaction, as product is not lost in the form of insoluble by-products. Additionally, solids may block the apparatus when the process is performed in flow. Thus, a reduction in the amount of solid formation is particularly advantageous when the process is performed under flow conditions.

The use of a supported homogeneous or heterogeneous catalyst also allows for easy separation of the catalyst from the reaction mixture. In embodiments, the catalyst is separated from the reaction mixture by filtration. This may be performed during the reaction (e.g. by passing the reagents through the catalyst during the reaction) or once the reaction is complete.

In embodiments, a supported homogeneous or heterogeneous catalyst may be suspended in the reaction mixture. In these embodiments, the supported homogeneous catalyst may be removed by filtration after the reaction is complete. In alternative embodiments, the supported homogenous or heterogeneous catalyst may be fixed in the location of the reaction of step ii) such that the reagents flow through the catalyst. For example, the supported homogeneous or heterogeneous catalyst may be used in a packed bed reactor, where a column in the reactor is packed with the supported homogeneous or heterogneous catalyst. During the reaction, the reagents and reaction products flow through the column, whilst the supported homogeneous or heterogeneous catalyst remains within the column.

In embodiments in which the supported homogeneous or heterogeneous catalyst is filtered from the reaction mixture after the reaction is complete, the filter should be smaller than the catalyst particle size to ensure effective removal of the catalyst.

Due to the ease of separation of the reaction mixture from the supported homogeneous or heterogeneous catalyst, the reaction of step ii) can be performed as either a batch reaction process or a flow process.

Following reaction in step ii), the oxamide, oxamate or oxalate may be further purified. This may be performed by any suitable means, such as by chromatographic separation or distillation. Preferably, such purification may be performed under flow conditions and thus the reaction of step ii) is performed, optionally filtered, and purified under flow conditions. The resulting product may then be introduced into the reaction site of step iii) under flow conditions. The reaction of step iii) may then be performed in batch or in flow conditions, as discussed further below.

The oxamide, oxamate or oxalate produced in step ii) may be introduced directly into the reaction site of step iii), preferably after removal of any catalyst. In embodiments, the reaction sites of steps ii) and iii) are connected, such that the product of step ii) is transferred into the reaction site of step iii) without being removed from the apparatus. This transfer may be as part of a batch reaction process or a flow reaction process. In preferred embodiments, step ii) is performed under flow conditions using a supported homogeneous or heterogeneous catalyst, following which the supported homogeneous or heterogeneous catalyst is removed by filtration. Preferably a heterogeneous catalyst is used. The product of step ii) is then introduced directly into the reaction site of step iii).

Though less preferred, it is also envisaged that the reaction of step ii) may be performed according to the above description using any CO source, and thus independently of step i), in order to obtain an oxamide, oxamate or oxalate. The oxamide, oxamate or oxalate of this reaction may be reduced according to step iii) below. It is not required that step ii) is performed under flow conditions, but preferably this reaction is performed under flow conditions. This reaction step may be combined with step iii), such that step ii) and iii) are performed independently of step i), and thus the source of CO is not limited. It is not required that either step ii) or iii) is performed under flow conditions, but one or both of these steps may be performed under flow conditions.

Step iii)

In the third step of the synthesis of EG, the oxamide, oxamate or oxalate from step ii) is reduced to produce EG.

The reduction in step iii) is preferably performed by catalytic hydrogenation, which has been found to result in high conversions. Any suitable conditions for such hydrogenation may be used and can be found in literature.

Preferably the catalyst used in the catalytic hydrogenation reaction comprises one or more metals from Groups VIII to XI, preferably a metal selected from silver, iron, ruthenium, rhodium, nickel, palladium, platinum and/or copper. This catalyst may be in the form of a metal complex. In embodiments, the catalyst is an iron or ruthenium metal complex. Alternatively, the catalyst may be a heterogeneous catalyst. The heterogenous catalyst may be supported, as described for the heterogeneous catalyst in step ii). For example, the catalyst may be Ag/Al₂O₃ or Ru/Al₂O₃.

In embodiments in which the catalyst is a metal complex, the ligands may be bound to the metal centre by a nitrogen, photphorous and/or oxygen atom. Such ligands include secondary or tertiary amines such as trimethylamine, triethylamine, a piperidine, 4-dimethylaminopyridine (DMAP), 1,4-diazabicyclo[2.2.2]octane (DABCO), proline, phenylalanine, a thiazolium salt, N-diisopropylethylamine (DIPEA or DIEA), bipyridyle (bipy), terpyridine (terpy), phenanthroline (phen), ethylenediamine, N,N,N′,N′-tetra-methyl-ethylenediamine (TMEDA), a quinoline and pyridine; alkyl and aryl phosphines such as triphenylphosphine, 2,2′-bis (diphenylphosphino)-1,1′-binaphthyle (BINAP), triisopropylphosphine, tris[2-diphenylephosphino)ethyl]phosphine (PP), tricyclohexylphosphine, 1,2-bis-diphenyphosphinoethane (dppe), 1,2-bis (diphenylphosphino)ethane (dppb); and alkyl and aryl phosphonates such as diphenylphosphate, triphenylphosphate (TPP), tri(isopropylphenyl)phosphate (TIPP), cresyldiphenyl phosphate (CDP), tricresylphosphate (TCP); alkyl and aryl phosphates such as di-nbutylphosphate (DBP), tris-(2-ethylhexyl)-phosphate and triethyl phosphate; oxygen bases such as acetate (OAc), acetylacetonate, methanolate, ethanolate, benzoyl peroxide; and mixed heteroatom ligands. For example, the catalyst maybe carbonylhydrido(tetrahydroborato)[bis(2-diphenylphosphinoethyl)amino]ruthenium(II) (sold under the trade name Ru-MACHO-BH®).

The catalyst may be present in an amount of at least 0.001 molar equivalents, preferably at least 0.005 molar equivalents, and more preferably at least 0.01 molar equivalents of the oxamide, oxamate or oxalate. The catalyst may be present in an amount of up to 0.1 molar equivalents, preferably up to 0.075 molar equivalents, and more preferably up to 0.05 molar equivalents of the oxamide, oxamate or oxalate. Thus, the catalyst may be present in an amount of from 0.001 to 0.1 molar equivalents, preferably from 0.005 to 0.075 molar equivalents, and more preferably from 0.01 to 0.05 molar equivalents of the oxamide, oxamate or oxalate.

The catalytic hydrogenation reaction is performed under an atmosphere comprising or consisting essentially of hydrogen. The atmosphere may comprise one or more inert gases, such as N₂ or Ar. Preferably, the atmosphere consists essentially of H₂. The H₂ may be obtained from step i) if the electrolyte comprises H₂O, as described in detail above. Preferably, H₂ is directly obtained from a dedicated electrolyser that generates H₂ from H₂O. Optionally, H₂ obtained from step i) may be combined with H₂ obtained from other sources, such as from a dedicated electrolyser for producing H₂.

The pressure of H₂ may be at least 1 MPa, preferably at least 2 MPa, and more preferably at least 4 MPa. The reaction may be performed at a H₂ pressure of up to 10 MPa, preferably up to 8 MPa, and more preferably up to 6 MPa. Thus, the reaction is preferably performed at a pressure of from 1 to 10 MPa, preferably from 2 to 8 MPa, and more preferably from 4 to 6 MPa.

The reaction is preferably performed at a temperature of at least 50° C., preferably at least 80° C., and more preferably at least 100° C. The reaction is preferably performed at a temperature of up to 200° C., preferably up to 175° C., and more preferably up to 150° C. Thus, the reaction is preferably performed at a temperature of from 50 to 200° C., preferably from 80 to 175° C., and more preferably from 100 to 150° C.

When the reaction is performed under batch conditions, the reaction may be performed for at least 30 minutes, preferably at least 1 hour, and more preferably at least 10 hours. The reaction may be performed for up to 72 hours, preferably up to 24 hours, and more preferably up to 16 hours. Thus, the reaction may be performed for a duration of from 10 minutes to 72 hours, preferably from 1 to 24 hours, and more preferably from 10 to 16 hours. The reaction may be considered complete once this time period has elapsed.

When the reaction is performed under flow conditions, the residence time is at least at least 30 seconds, preferably at least 1 minute, and more preferably at least 15 minutes. The residence time may be up to up to 5 hours, preferably up to 1 hour, and more preferably up to 30 minutes. Thus, the residence time may be performed for a duration of from 30 seconds to 5 hours, preferably from 1 minute to 1 hour, and more preferably from 15 to 30 minutes.

Conversion of the oxamide, oxamate or oxalate to EG is preferably at least 50%, more preferably at least 70%, even more preferably at least 80% and most preferably at least 90%.

The reaction may be passed through a series of reactors. Additionally or alternatively, the reaction mixture may be passed through the reactor in a loop. Thus, the system can achieve the desired conversions, preferably complete conversion, if this is not achieved initially.

Catalytic hydrogenation may be performed in the presence of a solvent system. The solvent system may comprise THF, DME, toluene, dioxane, water, ethanol and mixtures thereof. Preferably the solvent system is toluene or dioxane, preferably dioxane. If step ii) is performed in a different solvent system, preferably the solvent system of reaction in step ii) is removed and replaced by the desired solvent system.

Preferably, catalytic hydrogenation is performed under basic conditions. Any suitable base may be used. In embodiments an inorganic base is used, such as K₂CO₃, LiOH, NaOH, KOH, ^{t}BuOLi, ^{t}BuONa and/or ^{t}BuOK, preferably K₂CO₃, NaOH, KOH and/or ^{t}BuOK, more preferably K₂CO₃.

The base may be present in an amount of at least 0.01 molar equivalents, preferably at least 0.05 molar equivalents, and more preferably at least 0.08 molar equivalents of the oxamide or oxamate. The base may be present in an amount of up to 0.2 molar equivalents, preferably up to 0.15 molar equivalents, and more preferably up to 0.12 molar equivalents of the oxamide or oxamate. Thus, the base may be present in an amount of from 0.01 to 0.2 molar equivalents, preferably from 0.05 to 0.15 molar equivalents, and more preferably from 0.08 to 0.12 molar equivalents of the oxamide or oxamate.

Unless otherwise stated, the quantities of other reagents are measured against the quantity of the oxamide, oxamate or oxalate. Thus, the oxamide, oxamate or oxalate will be present in 1 molar equivalent.

Alternatively, reduction in step iii) may be performed by electroreduction.

This reduction reaction may be performed under batch or flow conditions. Preferably the reaction is performed under flow conditions.

In preferred embodiments, the reduction is a catalytic hydrogenation which is performed under flow conditions. In preferred embodiments, both step ii) and step iii) are performed under flow conditions.

The catalyst may be removed from the reaction mixture using any suitable technique. For example, the reaction mixture may be filtered, preferably through celite. Alternatively, if the reactor is a fixed bed reactor, the catalyst remains in the rector following removal of the reaction mixture from the reactor

Preferably, EG is separated from the reaction mixture by distillation. Distillation may be performed at atmospheric pressure or at reduced pressure. Distillation may be performed using standard conditions. Separation using distillation results in a high purity product and does not rely upon other factors such as phase separation, which can be influenced by the reaction conditions, such as the solvent used and the presence of salts.

The reaction may result in a biphasic reaction mixture, for example with one phase comprising the EG and the other phase the solvent and amine or alcohol by-product. In this situation, the product can be separated from the by-product and the catalyst using any known techniques, such as phase separation and/or filtration.

Amine and/or alcohol may be produced in step iii), depending upon the product of step ii). Preferably, the amine and/or alcohol produced in step iii) is recycled to step ii). The amine or alcohol may be separated from the other products in step iii) using known techniques, such as separation, distillation and/or filtration. Purification of the amine and/or alcohol may be performed if required.

Preferably the EG has a purity of at least 80%, preferably at least 90%, more preferably at least 95%, most preferably at least 99%. It has been found that EG having a purity of greater than 99.7% can been achieved using the process described herein.

EG produced using this method has been compared to commercial EG obtained from fossil fuels and has been found to have a similar purity. In particular, well known nuclear magnetic resonance (NMR) and gas chromatography-mass spectrometry (GC-MS) methods may be used to determine the purity of and to compare these EG products. These methods may be performed using conventional equipment under standard conditions. In both methods, the spectra were found to be identical, demonstrating that EG produced using this method may be used in place of commercially available EG.

Additionally, inductively coupled plasma-mass spectrometry (ICP-MS) may be performed in order to determine the metal content of the EG produced using this method. ICP-MS may be performed on an ICP-MS 7700x Agilent machine using a Multiwave Eco Anton PAAR microwave. The metal content was found to be very low, below ppm levels, indicating that the metal catalyst is effectively removed from the EG product during the reaction process.

Synthesis of PET

PET can be synthesised by performing an esterification reaction between terephthalic acid and EG, or by the transesterification reaction between EG and a terephthalate di-ester, such as dimethyl terephthalate. By using the EG produced in the above process, the production of PET will not rely on the use of fossil fuels and will result in a greener, more sustainable process.

It has been found that PET synthesised using EG produced using this method compared favourably to PET produced using the same process from commercial EG obtained from fossil fuels. For example, the polymers were found to have similar intrinsic viscosities, number average molecular weights, weight average molecular weights, glass transition temperatures and melting ranges. Thus, the EG produced according to the process described herein may be used in place of commercial EG sources to produce PET having the same or very similar properties.

In particular, it has been found that polymers obtained from both sources of EG can achieve very similar intrinsic viscosities when produced under the same conditions. The intrinsic viscosity may be measured using standard methods. For example, the intrinsic viscosity of the samples may be measured at 25° C. with an Ubbelohde-la viscometer in dichloroacetic acid (99%) according to DIN EN ISO 1628-5. The intrinsic viscosity is dependent on the length of the polymer chains, with longer chains resulting in more entanglements and thus a higher viscosity. Thus, the polymer produced from both sources of EG achieved similar average chain lengths.

Gel permeation chromatography (GPC) can be used to determine the molecular weight of polymers. GPC is a type of size exclusion chromatography (SEC) that separates polymers on the basis of size. GPC is a well-known technique and can be performed using conventional equipment under standard conditions. For example, GPC can be performed on an Agilent Technologies 1260 Infinity II High Temperature GPC System (GPC 220, Agilent Technologies, Inc, Santa Clara, USA) equipped with a refractive index detector and operated at 50° C. using m-cresol as eluent. PET standards with narrow molecular weight distributions can be used for calibration purposes. The number average molecular weight (Mₙ) and the weight average molecular weight (M_{w}) of the PET polymer chains can be determined using this technique. The polydispersity (PD) can be calculated from these values using the equation PD=M_{w}/Mₙ. Preferably, the PD value is low as this indicates a narrower range of change lengths and thus a uniform polymer. It has been found that PET formed from EG produced as described herein has very similar M_{w}, Mₙ and PD values to that produced using commercial EG.

Differential scanning calorimetry (DSC) analysis can be performed on polymers to determine the thermal properties of the polymer. DSC can be used to calculate the glass transition temperature (T_{g}) and the melting range of the polymer. DSC is a well-known technique and can be performed using conventional equipment under standard conditions. For example, DSC measurements may be carried out under air (20 mL/min) on a Q2000 differential scanning calorimeter (TA Instruments Inc., New Castle, Del., USA) while applying a heating rate of 10 K/min. The melt enthalpy ΔHₘ, melting peak temperature T_{m,p} and T_{g} can be determined from the heat flow-temperature curves. It has been found that both the T_{g} and the melting range of both PET polymers were identical.

### EXAMPLES

### Example 1—General Procedure for Conversion of CO₂ to CO in Step i)

An electrochemical cell containing an electrocatalyst is optionally linked to a purification module and gas mixer. During the reaction, CO₂ is introduced into the electrochemical cell and reduced by an electrocatalyst, to produce a mixture of CO and O₂. The gas mixture may optionally be purified in the purification module to remove unwanted gases e.g. to produce pure CO gas. The gas mixture from the electrochemical cell, or the gas produced from the purification module if present, may optionally be directed to the gas mixer to produce a mixture of CO:O₂ in a ratio of 2:1.

### Example 2—General Procedure for Oxidative Carbonylation of an Amine or Alcohol into an Oxamide in Step ii)

Batch Reaction:

A reactor, preferably an autoclave, is loaded with the desired catalyst (0.00001 to 0.1 molar equivalents), amine or alcohol (1 molar equivalent), optionally promotor (0.005 to 0.1 molar equivalents), optionally base (0.001 to 0.4 molar equivalents) and solvent (50 to 6000 mL per mole amine or alcohol). The autoclave is sealed and purged 4 times with 10 bar of the CO/O₂ mixture from step i), following which the temperature is maintained at between 25 and 200° C. for the duration of the reaction. The CO/O₂ pressure is maintained at between 1 and 100 bar for the duration of the reaction. The reaction is performed for a duration of from 0.1 to 72 hours. Once the reaction is complete, the autoclave is cooled to ambient temperature.

If a supported homogeneous catalyst or heterogeneous is used, these are separated from the reaction mixture by filtration.

Flow Reaction Using a Homogeneous Catalyst:

A solution of homogeneous catalyst (0.00001 to 0.1 molar equivalents), amine or alcohol (1 molar equivalent), optionally promotor (0.005 to 0.1 molar equivalents), optionally base (0.001 to 0.4 molar equivalents) and solvent (50 to 6000 mL per mole amine or alcohol) is prepared and pumped into a reactor. The temperature in the reactor is maintained at between 25 and 200° C., the CO/O₂ pressure is maintained at between 1 and 100 bar and the flow rate selected so as to provide a residence time of from 10 minutes to 4 hours.

The solution can be recirculated until the completion of the reaction.

Flow Reaction Using a Heterogeneous or Supported Homogeneous Catalyst:

A solution of amine or alcohol (1 molar equivalent), optionally promotor (0.005 to 0.1 molar equivalents), optionally base (0.001 to 0.4 molar equivalents) and solvent (50 to 6000 mL per mole amine or alcohol) is prepared and pumped into a reactor containing a heterogeneous or supported homogeneous catalyst (0.00001 to 0.1 molar equivalents), such as a packed bed reactor. The temperature in the reactor is maintained at between 25 and 200° C., the CO/O₂ pressure is maintained at between 1 and 100 bar and the flow rate selected so as to provide a residence time of from 10 minutes to 4 hours.

The solution can be recirculated until the completion of the reaction.

### Example 3—General Procedure for Reduction of the Oxamide to Ethylene Glycol (EG) in Step iii)

If the solvent of step ii) is also used in step iii), the crude reaction mixture is filtered through Celite (2-3 cm thick), and the filtrate added to an autoclave.

If the solvent of step ii) is not used in step iii), the solvent of the reaction mixture from step ii) is removed in vacuo, following which toluene is added to the product. The resulting mixture is filtered through Celite (2-3 cm thick), and the filtrate added to a reactor.

Batch Reaction:

The new solution in the reactor, which is preferably an autoclave, is loaded with the desired catalyst (0.001 to 0.1 molar equivalents), base (0.001 to 0.2 molar equivalents) and solvent. The reactor is sealed and purged 3 times with 10 bar of H₂, following which the reactor is pressurised to 50 bar H₂. The temperature is then maintained at between 30 and 200° C. and the reaction performed for a duration of from 10 minutes to 24 hours.

Flow Reaction:

The new solution in the reactor is loaded with the desired base (0.001 to 0.2 molar equivalents) and solvent. If the catalyst is a heterogeneous or supported homogeneous catalyst, the reactor is preferably a packed bed reactor including the catalyst (0.001 to 0.1 molar equivalents). Alternatively, the reactor may be loaded with catalyst (0.001 to 0.1 molar equivalents). The temperature in the reactor is maintained at between 30 and 200° C., the H₂ pressure is maintained at 50 bar H₂ and the flow rate selected so as to provide a residence time of from 10 minutes to 24 hours.

Purification:

Once the reaction is complete, solvent is removed under reduced pressure and the residue is distilled at a temperature of 120° C. and a pressure of from atmospheric pressure to 10 mbar in order to remove solvent residue and impurities, following which EG is distilled at a temperature of 120° C. and a pressure of from 10 mbar and 5 mbar to produce EG with a purity of >99%.

Alternatively, once the reaction is complete, the autoclave is cooled to ambient temperature, yielding a biphasic mixture (EG in one phase and solvent, amine or alcohol co-product, catalyst and any side products in another phase). Simple phase separation provided EG with a purity of >95%. The volatile compounds are removed under reduced pressure and the liquid containing the EG is purified by silica gel chromatography, producing analytically pure EG.

### Example 4—Step ii)—Oxidative Carbonylation of an Amine into an Oxamide According to Example 2

The oxidative carbonylation of an amine was performed according to Example 2 under batch reaction conditions using the supported homogeneous catalysts described in Table 1 (obtainable from Sigma Aldrich or SiliCycle).

The catalyst (0.03 molar equivalents) was introduced into an autoclave equipped with a 300 mL glass sleeve and THF (180 mL) was added. The solution was stirred for 10 min at room temperature, then ⁿBu₄NI (2.96 g, 8 mmol, 0.08 molar equivalents), K₂CO₃ (5.52 g, 40 mmol, 0.4 molar equivalents) and piperidine (10 mL, 100 mmol, 1 molar equivalent) were added. The autoclave was then loaded with 10 bar of O₂ and 25 bar of CO. The reaction was stirred for 16 h at room temperature before opening slowly the system to release the pressure. The solution was transferred to a 500 mL monocol, and the THF was evaporated under reduced pressure. The crude product was then dissolved in toluene (200 mL), and stirred for 30 minutes, before filtering the solution on silica gel (5 cm), using toluene as elution solvent. The recovered filtrate is concentrated under reduced pressure, in order to recover a bright yellow solution (approximately 120 mL), containing the desired oxamide in toluene.

The solution of oxamide in toluene was then used in the next step without further purification.

TABLE 1
Conversion of piperidine to oxamide using various catalysts.

| | Conversion of |
|---|---|
| Catalyst | piperidine (%) |
| Di(acetato)dicyclohexylphenylphosphinepalladium(II), | 90 |
| polymer-bound FibreCat ® | |
| Dichlorobis(triphenylphosphine)palladium(II), | 93 |
| polymer-bound | |
| Bis[(diphenylphosphanyl)methyl]amine palladium(II) | 81 |
| acetate, polymer-bound | |
| SiliaCat DPP-Pd Heterogeneous Catalyst (R390-100) | 55 |
| PEPPSI-IPr [PdCl₂(IPr)(3-Cl-py)] | 89 |

Example 5—Step iii)—Selective Hydrogenation of Oxamide to Ethylene Glycol According to Example 3

The 1,10-oxalyl dipiperidine (1 mmol) produced in Example 4 was reduced to ethylene glycol according to Example 3 using batch reaction conditions under the following conditions (see Table 2).

The solution of oxamide in toluene from Example 4 was introduced into a 250 ml monocol and the reaction mixture purged with argon before introducing the catalyst (see Table 2) and the base (see Table 2), under an inert atmosphere. The solution was stirred for 15 min, then transferred to the glass sleeve of the autoclave, fitted with a straight bar. The autoclave was then purged 3 times with 15 bar of hydrogen, then charged with 46 bar of hydrogen, and allowed to heat to 160° C. with stirring. Due to the heating, the system increased in pressure and stabilised at around 70 bar after one hour. The reaction was stirred for 16 h under these conditions, after which the autoclave was left cool to room temperature, and the system is opened slowly to release the hydrogen. The reaction mixture was transferred to a 250 mL monocol, and distilled under atmospheric pressure to recover a colourless solution containing ethylene glycol in toluene.

TABLE 2
Conditions for the reduction of 1,10-oxalyl dipiperidine.

| | | Temper- | Dura- | Conver- |
|---|---|---|---|---|
| | | ature | tion | sion |
| Catalyst | Base | (°C) | (h) | (%) |
| 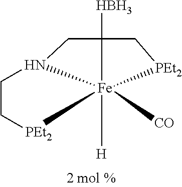 | KOH 5 mol % | 160 | 16 | 69 |
| 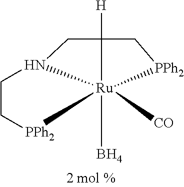 | NaOH 2 mol % | 160 | 16 | 99 |
| 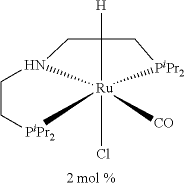 | ^{t}BuOK 10 mol % | 160 | 16 | 90 |

### Example 6—Step ii) Oxidative Carbonylation of an Amine into an Oxamide Under Batch and Flow Conditions

The following reactions were performed using the catalysts listed in Table 3. The catalyst equivalents are molar equivalents calculated based on the amine (i.e. amine=1 molar equivalent). The promotor was used in an amount of 2.5 mol % of the amine (0.025 molar equivalents) and the solvent was used in an amount of 600 to 6000 mL per mole of amine.

TABLE 3
Catalysts

| Number | Catalyst |
|---|---|
| 1 | Pd(acac)₂ |
| 2 | Ni(CO)₄ |
| 3 | Fe₃CO₁₂ |
| 4 | Pd/γ-Al₂O₃ calcinated |
| 5 | Pd/C |
| 6 | Ag/γ-Al₂O₃ calcinated |
| 7 | Co(5 wt %)/SiO₂ calcinated |
| 8 | Co(5 wt %)/γ-Al₂O₃ calcinated |
| 9 | Fe(5 wt %)/γ-Al₂O₃ calcinated |
| 10 | Ni/γ-Al₂O₃ calcinated |

The oxidative carbonylation of an amine was performed under batch conditions using a homogeneous catalyst. These reactions were performed under the following conditions and resulted in the following conversion of amine to oxamide (see Table 4). The batch reactor used was a Parr industry reactor or a Berghof reactor.

TABLE 4
Batch process using a homogeneous catalyst

| | Cat. | | | | T | O₂:CO | Time | |
|---|---|---|---|---|---|---|---|---|
| Cat. | (equiv.) | Amine | Promotor | Solvent | (° C.) | (bar) | (h) | Conversion |
| 1 | 0.0001 to | Pyrrolidine | nBu₄NI | Dioxane | 25 | 10:40 | 16 h | 99% |
| | 0.01* | | | | | | | |
| 2 | 0.01 | Pyrrolidine | nBu₄NI | CH₃CN | 80 | 10:40 | 25 | 76% |
| 3 | 0.01 | Pyrrolidine | nBu₄NI | CH₃CN | 80 | 10:40 | 16 | 80% |

*This reaction was performed multiple times under the conditions listed above with catalyst equivalents in the range of from 0.001 to 0.01 with respect to the amine. All experiments resulted in the same conversion.

This reaction was also performed under conditions analogous to those used in Dong, K., Elangovan, S., Sang, R. et al. Selective catalytic two-step process for ethylene glycol from carbon monoxide. Nat Commun 7, 12075 (2016). Namely, a reaction similar to the reaction in row 1 of Table 4 was performed in the presence of tri(o-tolyl)phosphine and K₂CO₃. In this instance, the yield was the same as that of the reaction performed according to the present invention, but solid formation was observed. In particular, solid K₂CO₃ was observed and Pd(0) was precipitated during the reaction. In the reactions performed in Dong et al., degradation of the phosphine and the production of phosphine oxide is observed, which is undesirable as this makes purification of the oxamide difficult. Conversely, when the reaction was performed under the conditions disclosed herein, no phosphine oxide was produced whilst still attaining the same yield. In addition, the reactions performed in Dong et al. required long reaction durations of 64 hours, whereas as shown in Table 4, row 1, the same conversions were observed after 16 hours using the conditions of the present invention.

The oxidative carbonylation of an amine was performed under batch conditions using a heterogenous catalyst. These reactions were performed under the following conditions and resulted in the following conversion of amine to oxamide (see Table 5).

TABLE 5
Batch process using a heterogeneous catalyst

| | Cat. | | | | T | O₂:CO | Time | |
|---|---|---|---|---|---|---|---|---|
| Cat. | (equiv.) | Amine | Promotor | Solvent | (° C.) | (bar) | (h) | Conversion |
| 4 | 0.05 | Pyrrolidine | nBu₄NI | THF | 50 | 10:40 | 16 | 82% |
| 5 | 0.025 | Piperidine | nBu₄NI | THF | 25 | 10:40 | 16 | 99% |
| 6 | 0.05 | Pyrrolidine | nBu₄NI | Dioxane | 100 | 10:40 | 16 | 53% |
| 7 | 0.05 | Pyrrolidine | nBu₄NI | CH₃CN | 80 | 10:40 | 25 | 59% |
| 8 | 0.05 | Pyrrolidine | nBu₄NI | CH₃CN | 80 | 10:40 | 25 | 76% |
| 9 | 0.05 | Pyrrolidine | nBu₄NI | CH₃CN | 80 | 10:40 | 16 | 80% |
| 10 | 0.05 | Pyrrolidine | nBU₄NI | CH₃CN | 80 | 10:40 | 16 | 80% |

The oxidative carbonylation of an amine was performed under flow conditions using a homogeneous catalyst. These reactions were performed under the following conditions and resulted in the following conversion of amine to oxamide (see Table 6). All reactions were performed at 25° C.

TABLE 6
Flow process using homogeneous catalyst. All reactions were performed at 25° C.

| | Cat. | | | | O₂:CO | Residence | |
|---|---|---|---|---|---|---|---|
| Cat. | (equiv.) | Amine | Promotor | Solvent | (bar) | Time (min) | Conversion |
| 1 | 0.001 | Pyrrolidine | nBu₄NI | Dioxane | 10:40 | 16 | 99% |
| 1 | 0.01 | Pyrrolidine | nBu₄NI | Dioxane | 10:40 | 16 | 99% |
| 1 | 0.01 | Pipepridine | nBu₄NI | THF | 10:40 | 16 | 99% |
| Comparative* | 0.01 | Piperidine | nBu₄NI*** | THF | 25:25 | 16 | 75% |
| 1 + P(o-tol)₃** | | | | | | | |

*Comparative example with conditions analogous to those used in Dong, K., Elangovan, S., Sang, R. et al. Selective catalytic two-step process for ethylene glycol from carbon monoxide. Nat Commun 7, 12075 (2016). 10 mol % (0.1 molar equivalents) K₂CO₃ was used.
**P(o-tol)₃ = tri(o-tolyl)phosphine, 0.4 mol % P(o-tol)₃ with reference to piperidine.
***2 mol % (0.02 molar equivalents) nBu₄NI.

The oxidative carbonylation of an amine was performed under flow conditions using a heterogeneous catalyst. These reactions were performed under the following conditions and resulted in the following conversion of amine to oxamide (see Table 7).

TABLE 7
Flow process using heterogeneous catalyst

| | Cat. | | | | T | O₂:CO | Residence | |
|---|---|---|---|---|---|---|---|---|
| Cat. | (equiv.) | Amine | Promotor | Solvent | (° C.) | (bar) | Time (min) | Conversion |
| 5 | 0.025 | Piperidine | nBu₄NI | THF | 25 | 10:40 | 15 | 99% |
| 6 | 0.05 | Pyrrolidine | nBu₄NI | Dioxane | 100 | 10:40 | 30 | 90% |
| 7 | 0.05 | Pyrrolidine | nBu₄NI | CH₃CN | 80 | 10:40 | 30 | 76% |
| 9 | 0.05 | Pyrrolidine | nBu₄NI | CH₃CN | 80 | 10:40 | 30 | 80% |
| 10 | 0.03 | Pyrrolidine | nBu₄NI | THF | 80 | 10:40 | 30 | 60% |
| 10 | 0.05 | Pyrrolidine | nBu₄NI | CH₃CN | 80 | 10:40 | 30 | 80% |

These results demonstrate that catalysts comprising Ag, Fe, Co, Ni, Ru and Pd can catalyse the reaction in step ii) when used in catalytic amounts. Additionally, these results demonstrate that under these conditions, good yields can be obtained in both batch and flow processes.

The good yields observed in these examples, in particular when the reactions are performed in flow and under the conditions used in Tables 6 and 7, are the result of reduced solid formation when compared to reactions performed under conditions outside the ranges described herein. When conditions outside those described herein are used, solid formation occurs, resulting in reduced yields. This is exemplified by the comparative example shown in Table 6, in which a phosphine ligand is present in the catalyst and a base is used. Under these reaction conditions, significant solid formation was observed. In addition, as can be seen in Table 6, this reaction was performed under very similar reaction conditions to the other reactions performed according to the present invention, but the yield is significantly reduced. In particular, it was found that a large amount of the K₂CO₃ used in the comparative example was not dissolved. Thus, following the reaction, purification was required in order to remove the solid before further processing was performed.

It was found that solid formation in this reaction can result from deposition of the catalyst. For example, Pd-catalysts were lost through precipitation of Pd-black. Thus, without being bound by theory, it is believed that the reduction in yield may at least in part be the result of precipitation of the catalyst during the reaction. Solid deposition was also found to reduce the heat transfer coefficient, reducing the reactor temperature and thus detrimentally effected the efficiency of the reaction. Therefore, the good yields observed when the reaction is performed under the conditions taught herein are believed to result from a reduction of catalyst deposition and/or improved heat transfer.

Moreover, solid formation was found to result in pressure losses, which result in a reduction in pumping efficiency and/or plugging, particularly when using flow conditions. Thus, the formation of solid also detrimentally affects the ability of the reaction to be performed under flow conditions. Therefore, reduction of solid formation when the reaction is performed under the conditions disclosed herein allows for the reaction to be performed effectively under flow conditions.

### Example 7—Step ii) Oxidative Carbonylation of an Alcohol into an Oxalate Under Batch and Flow Conditions

The following reactions were performed using the catalysts listed in Table 8 (some of which are reproduced from Table 3). The catalyst equivalents are molar equivalents calculated based on the alcohol (i.e. alcohol=1 molar equivalent). The promotor was used in an amount of 2.5 mol % of the alcohol (0.025 molar equivalents) and the solvent was used in an amount of 30 mL.

TABLE 8
Catalysts

| Number | Catalyst |
|---|---|
| 1 | Pd(acac) |
| 4 | Pd/γ-Al₂O₃ calcinated |
| 5 | Pd/C |
| 11 | Pd(OAc) |

The oxidative carbonylation of an alcohol was performed under batch conditions using a homogeneous or heterogeneous catalyst. These reactions were performed under the following conditions and resulted in the following conversion of alcohol to oxalate (see Table 9). The batch reactor used was a Parr industry reactor or a Berghof reactor.

TABLE 9
Batch process

| | Cat. | | | | T | O₂:CO | Time | |
|---|---|---|---|---|---|---|---|---|
| Cat. | (equiv.) | Alcohol | Promotor | Solvent | (° C.) | (bar) | (h) | Conversion |
| 1 | 0.03 | EtOH | nBu₄NI | THF | 100 | 10:40 | 16 | 60% |
| 5 | 0.10 | EtOH | nBu₄NI | CH₃CN | 100 | 10:40 | 16 | 76% |
| 11 | 0.05 | EtOH | nBu₄NI | CH₃CN | 100 | 10:40 | 16 | 80% |
| 4 | 0.10 | EtOH | nBu₄NI | THF | 100 | 10:40 | 16 | 60% |

The oxidative carbonylation of an alcohol was performed under flow conditions using a homogeneous or heterogeneous catalyst. These reactions were performed under the following conditions and resulted in the following conversion of alcohol to oxalate (see Table 10).

TABLE 10
Flow process

| | Cat. | | | | T | O₂:CO | Residence | |
|---|---|---|---|---|---|---|---|---|
| Cat. | (equiv.) | Alcohol | Promotor | Solvent | (° C.) | (bar) | Time (min) | Conversion |
| 1 | 0.03 | EtOH | nBu₄NI | THF | 100 | 10:40 | 30 | 75% |
| 5 | 0.05 | EtOH | nBu₄NI | CH₃CN | 100 | 10:40 | 30 | 84% |
| 11 | 0.05 | EtOH | nBu₄NI | CH₃CN | 100 | 10:40 | 30 | 89% |
| 4 | 0.03 | EtOH | nBu₄NI | THF | 100 | 10:40 | 30 | 74% |

The good yields observed in these examples, in particular when the reactions are performed in flow and under the conditions used in Table 10, are the result of reduced solid formation when compared to reactions performed under conditions outside the ranges described herein. When conditions outside those described herein are used, solid formation occurs, resulting in reduced yields.

As discussed in Example 6, the good yields observed when the reaction is performed under the conditions taught herein are believed to result from a reduction of catalyst deposition and/or improved heat transfer. Moreover, solid formation was found to result in pressure losses, which result in a reduction in pumping efficiency and/or plugging, particularly when using flow conditions. Therefore, reduction of solid formation when the reaction is performed under the conditions disclosed herein allows for the reaction to be performed effectively under flow conditions.

### Example 8—Step iii)—Selective Hydrogenation of Oxamide to Ethylene Glycol

The following reactions were performed using the catalysts listed in Table 11. The catalyst equivalents are molar equivalents calculated based on the oxamide (i.e. oxamide=1 molar equivalent).

TABLE 11
Catalysts

| Number | Catalyst |
|---|---|
| 12 | Ag/γ-Al₂O₃ calcinated |
| 13 | carbonylhydrido(tetrahydroborato)[bis(2- |
| | diphenylphosphinoethyl)amino]ruthenium(II) |
| | (Ru-MACHO-BH ®) |
| 14 | Ru/γ-Al₂O₃ calcinated |

Oxamide hydrogenation was performed under batch conditions using a heterogeneous catalyst. These reactions were performed under the following conditions and resulted in the following conversion of oxamide to EG (see Table 12).

TABLE 12
Batch process using heterogeneous catalyst

| | Cat. | | | | T | H₂ | Time | |
|---|---|---|---|---|---|---|---|---|
| Cat. | (equiv.) | Oxamide | Base | Solvent | (° C.) | (bar) | (h) | Conversion |
| 12 | 0.05 | Diphenyloxalamide | tBuOK | Dioxane | 150 | 70 | 16 | 70% |
| | | | (5 mol %) | | | | | |

Oxamide hydrogenation was performed under batch conditions using a homogeneous catalyst. These reactions were performed under the following conditions and resulted in the following conversion of oxamide to EG (see Table 13).

TABLE 13
Batch process using homogeneous catalyst

| | Cat. | | | | T | H₂ | Time | |
|---|---|---|---|---|---|---|---|---|
| Cat. | (equiv.) | Oxamide | Base | Solvent | (° C.) | (bar) | (h) | Conversion |
| 13 | 0.01 | Diphenyloxalamide | tBuOK | Toluene | 150 | 60 | 16 | >99% |
| | | | (3 mol %) | | | | | |
| 13 | 0.01 | 1,2-dipyrrolidin-1- | tBuOK | Toluene | 150 | 60 | 16 | 89% |
| | | ylethane-1,2-dione | (3 mol %) | | | | | |

Oxamide hydrogenation was performed under flow conditions using a homogenous catalyst. These reactions were performed under the following conditions and resulted in the following conversion of oxamide to EG (see Table 14).

TABLE 14
Flow process using homogeneous catalyst

| | Cat. | | | | T | H₂ | Time | |
|---|---|---|---|---|---|---|---|---|
| Cat. | (equiv.) | Oxamide | Base | Solvent | (° C.) | (bar) | (h) | Conversion |
| 13 | 0.01 | 1,2-dipyrrolidin-1- | tBuOK | Toluene | 150 | 60 | 20 | 96% |
| | | ylethane-1,2-dione | (2 mol %) | | | | | |
| 13 | 0.01 | 1,2-dipyrrolidin-1- | tBuOK | THF | 150 | 60 | 20 | 88% |
| | | ylethane-1,2-dione | (5 mol %) | | | | | |

Oxamide hydrogenation was performed under flow conditions using a heterogenous catalyst. These reactions were performed under the following conditions and resulted in the following conversion of oxamide to EG (see Table 15).

TABLE 15
Flow process using heterogeneous catalyst

| | Cat. | | | | T | H₂ | Residence | |
|---|---|---|---|---|---|---|---|---|
| Cat. | (equiv.) | Oxamide | Base | Solvent | (° C.) | (bar) | time (min) | Conversion |
| 12 | 0.05 | 1,2-dipyrrolidin-1- | tBuOK | Dioxane | 150 | 60 | 30 | 52% |
| | | ylethane-1,2-dione | (2 mol %) | | | | | |
| 14 | 0.05 | 1,2-dipyrrolidin-1- | K₂CO₃ | Dioxane | 100 | 60 | 30 | 39% |
| | | ylethane-1,2-dione | (2 mol %) | | | | | |
| 14 | 0.05 | 1,2-dipyrrolidin-1- | K₂CO₃ | THF | 100 | 60 | 30 | 60% |
| | | ylethane-1,2-dione | (2 mol %) | | | | | |

Thus, it has been demonstrated that hydrogenation of an oxamide to EG can be achieved using catalysts comprising Ru and Ag, using homogeneous and heterogeneous catalysts and in both batch and flow processes.

The use of heterogeneous catalysts is particularly beneficial as they have been found to be easier to synthetise, purify and reuse. In addition, heterogeneous catalysts have been found to be more stable and cheaper than homogeneous catalysts.

Additionally, the data in Tables 14 and 15 demonstrate that even using short reaction times, hydrogenation is observed. These conversions could be easily increased by, for example, extending the reaction time (e.g. the residence time for flow processes). However, preferably, a loop can be implemented to allow the system to get full conversion.

### Example 9—Step iii)—Selective Hydrogenation of Oxalate to Ethylene Glycol

The following reactions were performed using the catalysts listed in Table 11 (see Example 8). The catalyst equivalents are molar equivalents calculated based on the oxalate (i.e. oxalate=1 molar equivalent).

Oxalate hydrogenation was performed under batch conditions using a homogeneous or heterogeneous catalyst. These reactions were performed under the following conditions and resulted in the following conversion of oxalate to EG (see Table 16).

TABLE 16
Batch process

| | Cat. | | | | T | H₂ | Time | |
|---|---|---|---|---|---|---|---|---|
| Cat. | (equiv.) | Alkyl oxalate | Base | Solvent | (° C.) | (bar) | (h) | Conversion |
| 13 | 0.025 | Diethyloxalate | 7 mol % | THF | 120 | 60 | 16 | 99% |
| | | | tBuOK | | | | | |
| 13 | 0.003 | Diethyloxalate | 3 mol % | EtOH | 100 | 90 | 4 | 99% |
| | | | tBuOK | | | | | |
| 13 | 0.003 | Dimethyloxalate | 3 mol % | EtOH | 100 | 90 | 7 | 99% |
| | | | tBuOK | | | | | |
| 14 | 0.05 | Diethyloxalate | N/A | Dioxane | 140 | 60 | 16 | 90% |
| 12 | 0.05 | Diethyloxalate | N/A | Dioxane | 140 | 60 | 16 | 72% |

Oxalate hydrogenation was performed under flow conditions using a homogeneous or heterogeneous catalyst. These reactions were performed under the following conditions and resulted in the following conversion of oxalate to EG (see Table 17).

TABLE 17
Flow process

| | Cat. | | | | T | H₂ | Residence | |
|---|---|---|---|---|---|---|---|---|
| Cat. | (equiv.) | Alkyloxalate | Base | Solvent | (° C.) | (bar) | Time (min) | Conversion |
| 13 | 0.025 | Diethyloxalate | 3 mol % | THF | 120 | 60 | 15 | 99% |
| | | | tBuOK | | | | | |
| 13 | 0.01 | Diethyloxalate | 3 mol % | EtOH | 120 | 60 | 15 | 99% |
| | | | tBuOK | | | | | |
| 14 | 0.05 | Diethyloxalate | N/A | Dioxane | 140 | 60 | 30 | 90% |
| 12 | 0.05 | Diethyloxalate | N/A | Dioxane | 140 | 60 | 30 | 80% |

Thus, it has been demonstrated that hydrogenation of an oxamide to EG can be achieved using catalysts comprising Ru and Ag, using homogeneous and heterogeneous catalysts and in both batch and flow processes. Moreover, flow processes were found to yield conversions similar to those achieved in flow, and heterogeneous catalysts were found to achieve similar conversions to heterogeneous catalysts.

### Example 10—EG Characterisation and Comparison to Commercial EG

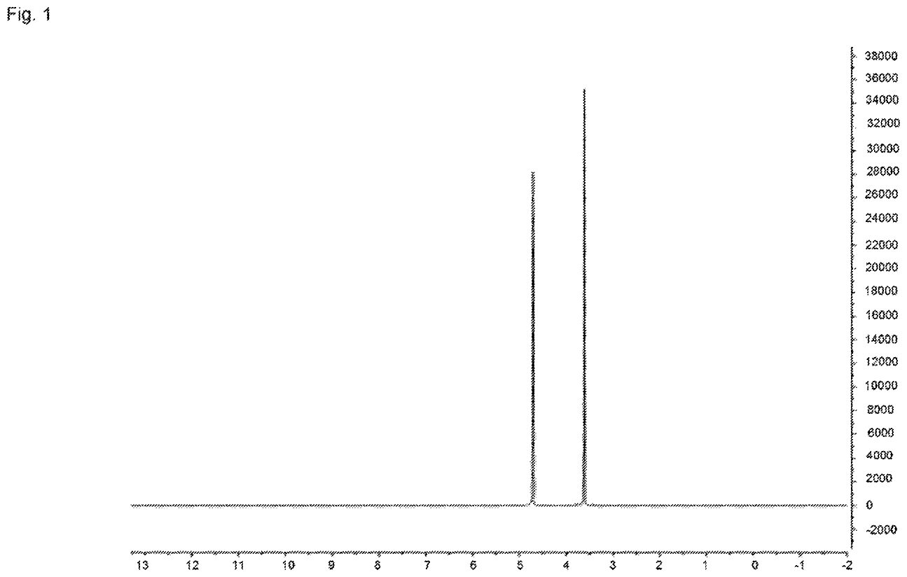

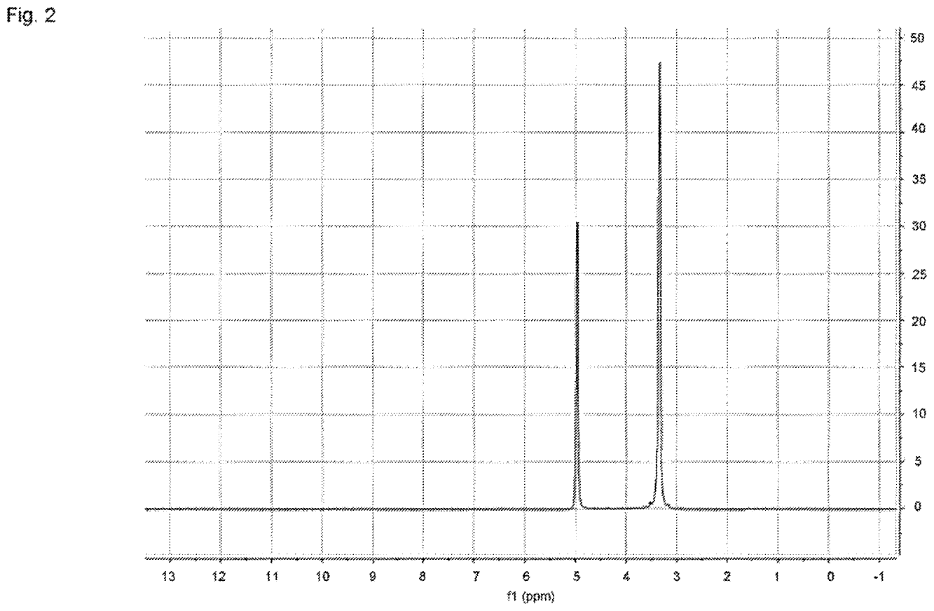

EG was produced according to Examples 1 to 3 using batch conditions for all steps and using a Pd(acac)₂ catalyst (i.e. catalyst 1 in Table 3) in THF in step ii) (for example see Examples 6 and 7) and a Ru-MACHO-BH®)catalyst (i.e. catalyst 13 in Table 11) in toluene in step iii) (for example see Example 8). The EG produced was analysed by ¹H NMR in D₂O and the resulting spectrum is shown in FIG. 1. Commercial EG having a purity of 99.7% was analysed in the same manner and the resulting spectrum is shown in FIG. 2.

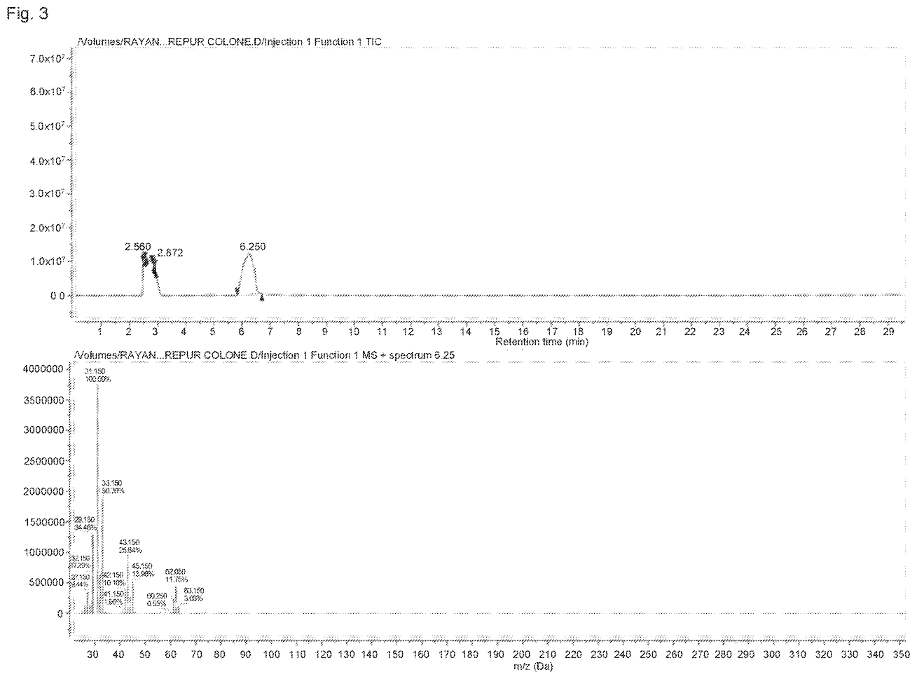

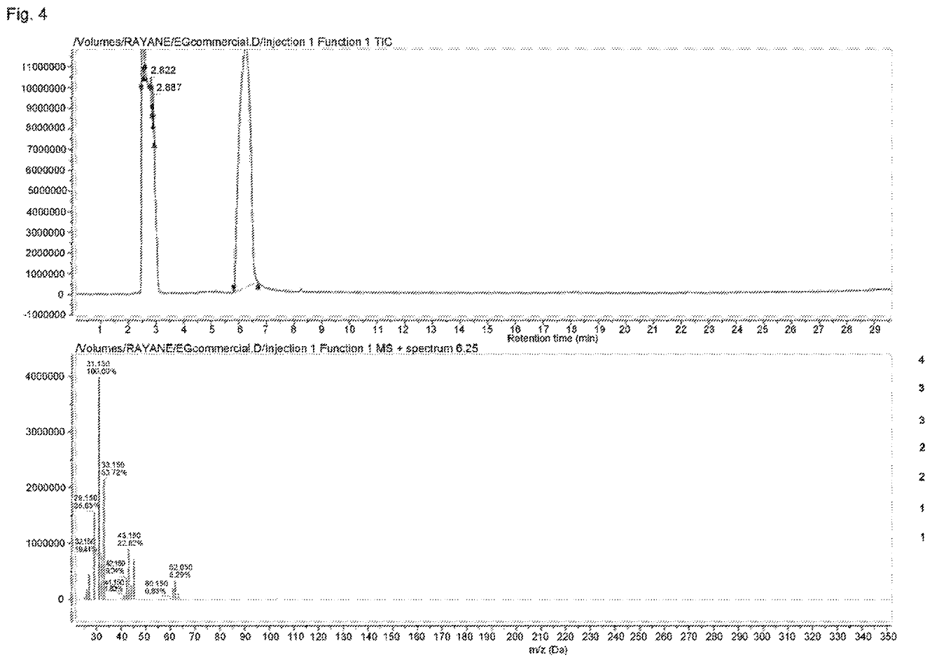

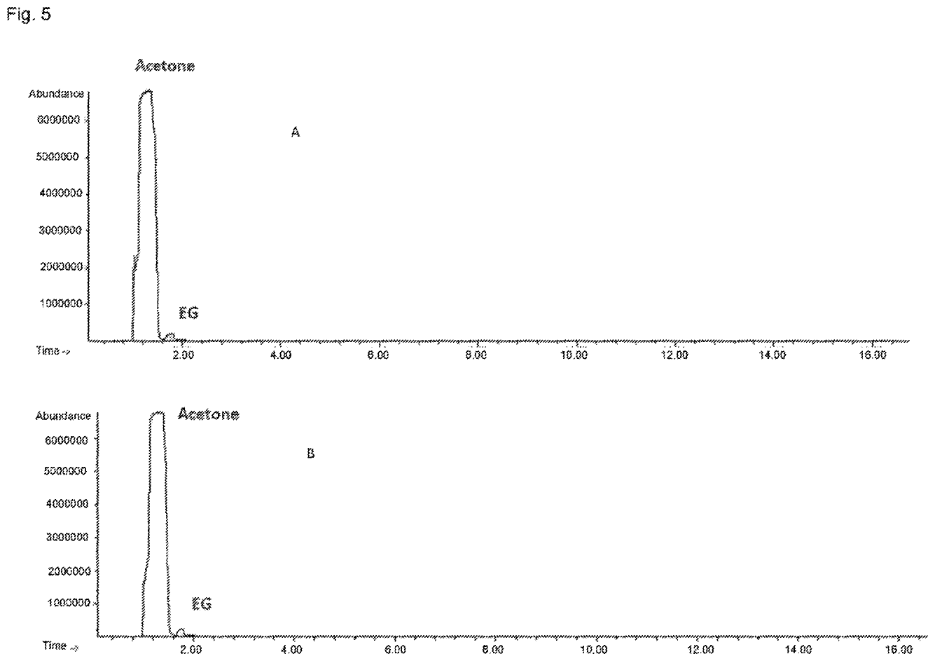

These samples were also analysed by GC-MS, with the spectra shown in FIGS. 3 and 5A for EG produced in the present method and FIGS. 4 and 5B for commercial EG. The mass spectrum shown in FIGS. 3 and 4 is of the product present in the peak in the GC spectrum having a retention time of about 6 to 7 minutes and corresponds to the mass spectrum expected for EG. In FIGS. 5A and B, EG is present in the smaller peak observed at just below 2 minutes, as confirmed by MS.

Both EG samples show the same NMR and GC-MS spectra, indicating that both samples have a similar composition and purity.

Finally, the EG produced using the present method was analysed using ICP-MS to determine the ruthenium and palladium metal content. As this sample was produced using a palladium catalyst in step ii) and a ruthenium catalyst in step iii), the metal content determined provides information on the amount of catalyst remaining in the product from these steps.

To prepare the EG samples for ICP-MS, 5 mg of EG was combined with 1 mL of 67% nitric acid, was placed in a microwave for 40 minutes, following which the mixture was diluted with a 3% nitric acid solution (50 mL).

ICP-MS was performed on an ICP-MS 7700x Agilent machine using a Multiwave Eco Anton PAAR microwave. Measurements were taken after mineralisation.

TABLE 18

| Ru (ppm) | Pd (ppm) |
|---|---|
| <0.02 | 0.88 |

The results of ICP-MS are shown in Table 18 above. These results demonstrate that low levels of catalyst, below one ppm, remain in the ethylene glycol produced according to the process of the present invention. No other metals were detected.

### Example 11—PET Synthesis and Analysis

PET was prepared via a transesterification reaction between EG and dimethyl terephthalate (DMT) using EG produced according to the present invention and commercial EG obtained from fossil fuels.

EG and DMT were combined in a ratio of 2.2:1 in the presence of manganese acetate as esterification catalyst. Sb₂O₃ catalyst (600 ppm) was then added, and the reaction performed for 4 days.

The intrinsic viscosity of the resulting polymers was measured at 25° C. with an Ubbelohde la viscometer in dichloroacetic acid (99%) according to DIN EN ISO 1628-5 for PET.

The M_{w} and Mₙ were measured for both PET polymers using GPC. An Agilent Technologies 1260 Infinity II High Temperature GPC System (GPC 220, Agilent Technologies, Inc, Santa Clara, USA) equipped with a refractive index detector was used and operated at 50° C. using m-cresol as eluent. Twenty milligrams of the PET sample was dissolved in a 20 mL m-cresol solution at 80 to 120° C. for 0.5 to 3 hours. Three consecutive PLgel Olexis columns (0.013 Å pore size) and one precolumn were used while applying a flow rate of 0.4 mL/min. For the recording and evaluation of the chromatograms, the GPC/SEC software of Agilent Technologies (Santa Clara, USA) was used. Narrow PET standards with 3,470 g/mol<Mw<115,000 g/mol were used for calibration. PD was calculated using the equation PD=M_{w}/Mₙ.

Additionally, the T_{g} and melting range of the resulting polymers was measured using DSC. DSC measurements were carried out under air (20 mL/min) on a Q2000 differential scanning calorimeter (TA Instruments Inc., New Castle, Del., USA) while applying a heating rate of 10 K/min. A sample mass of 2 mg was used. The melt enthalpy ΔHm and melting peak temperature Tm,p were determined from the heat flow-temperature curves, as well as the glass-transition temperature Tg using known methods.

These values are sown in Table 19 below.

TABLE 19
PET properties

| | | EG from present |
|---|---|---|
| EG source | Commercial EG | invention |
| Intrinsic viscosity of PET | 0.679 | 0.685 |
| produced from EG (cm³/g) | | |
| M_{w} (g/mol) | 104 | 93.0 |
| Mₙ (g/mol) | 32.8 | 35.8 |
| PD | 3.1 | 2.6 |
| T_{g} (° C.) | 80 | 80 |
| Melting range (° C.) | 230 to 260 | 230 to 260 |

As shown in Table 18, the properties of PET produced using EG produced according to the process described herein and PET produced using commercial EG resulted in very similar values for all parameters measured.

In particular, it is noted that the PET produced using EG synthesised according to the process described herein has a lower PD, which represents an improvement over PET produced using commercial EG.

Thus, it is evident that EG from the present invention can be used to produce PET of at least the same quality as that produced using commercially available PET.

1-26. (canceled)

27. A process for the production of ethylene glycol from CO₂, comprising the steps of:
i) reducing CO₂ to CO;
ii) reacting the CO produced in step i) with an amine to form an oxamide or an oxamate or with an alcohol to form an oxalate; and
iii) reducing the oxamide, oxamate or oxalate formed in step ii) to formethylene glycol.

28. The process according to claim 27, wherein the CO produced in step i) is introduced directly into step ii), and/or wherein the oxamide, oxamate or oxalate produced in step ii) is introduced directly into step iii).

29. The process according to claim 27, wherein step ii) is performed under flow conditions, preferably wherein steps i) and ii) or steps ii) and iii) are performed under flow conditions, more preferably wherein steps i), ii) and iii) are performed under flow conditions.

30. The process according to claim 27, wherein the reduction in step i) is electrochemical reduction, optionally wherein the electrochemical reduction in step i) is carried out in the presence of a porphyrin and/or a phthalocyanine catalyst, preferably a CoPc2 catalyst of Formula I or a FeTPP catalyst of Formula II:

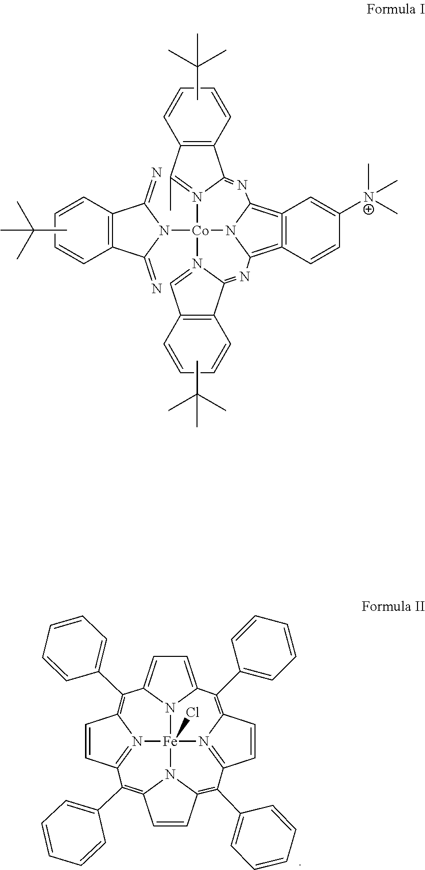

31. The process according to claim 30, wherein step i) is carried out in the presence of H₂O and the H₂O is reduced to produce H₂, and wherein the H₂ is preferably recycled for use in step iii).

32. The process according to claim 27, wherein the CO produced in step i) is purified prior to the reaction in step ii).

33. The process according to claim 27, wherein the reaction of step ii) is performed in the presence of a supported homogeneous catalyst, preferably wherein the supported homogeneous catalyst comprises a support and a metal centre coordinated to one or more ligands, wherein the metal centre is bound to the support via one or more of the ligands and wherein the ligand which is bound to the support comprises a phosphorous atom which is bound to the metal centre and/or wherein the metal centre is palladium and/or wherein the support comprises silica, alumina, a transition metal oxide, a polymer, carbon or mixtures thereof.

34. The process according to claim 27, wherein the reaction of step ii) is performed in the presence of a heterogeneous catalyst, preferably wherein the heterogeneous catalyst comprises a metal supported on a solid support, in particular wherein the metal is a Group VIII to XI metal and/or wherein the support comprises silica, alumina, a transition metal oxide, a polymer, carbon or mixtures thereof.

35. The process according to claim 27, wherein the reaction of step ii) is performed in the presence of a homogeneous catalyst, preferably wherein the homogeneous catalyst comprises a metal centre coordinated to one or more ligands.

36. The process according to claim 27, wherein in step ii) the molar ratio of CO in the atmosphere to amine or alcohol is from 2:1 to 12:1, preferably from 3:1 to 10:1, and more preferably from 4:1 to 9:1

37. The process according to claim 27, wherein step ii) is performed at a temperature of from 25 to 150° C., preferably from 30 to 110° C., and more preferably from 40 to 100° C.

38. The process according to claim 27, wherein step ii) is carried out in the presence of a solvent system, preferably wherein the solvent system comprises THF, toluene, acetonitrile or dioxane.

39. The process according to claim 27, wherein step ii) is carried out in the presence of a promotor, preferably wherein the promotor comprises iodine, an iodide derivative, an ammonium salt, or combinations thereof.

40. The process according to claim 39, wherein the promotor is present in an amount of from 0.005 to 0.075 molar equivalents, preferably from 0.01 to 0.05 molar equivalents, and more preferably from 0.02 to 0.03 molar equivalents of the amine or alcohol.

41. The process according to claim 27, wherein step ii) is performed under one or more of the following conditions:
in the presence of 0.00001 to 0.05 molar equivalents of catalyst with respect to the amine or alcohol;
under an atmosphere comprising CO and 02 in a molar ratio of from 3.5:1 to 4.5:1;
at a pressure of from 2 to 6 MPa;
at a temperature of from 25 to 100° C.;
in the presence of 100 to 900 mL of solvent system per mole of amine or alcohol;
in the presence of from 0.02 to 0.03 molar equivalents of promotor with respect to the amine or alcohol; and
the reaction is performed for a duration of from 6 to 25 hours if the reaction is performed under batch conditions, or the residence time is from 15 to 35 minutes if the reaction is performed under flow conditions,
preferably wherein step ii) is performed under all of these conditions.

42. The process according to claim 27, wherein reduction of step ii) is catalytic hydrogenation.

43. The process according to claim 42, wherein the catalyst used in the catalytic hydrogenation of step iii) comprises one or more metals from Groups VIII to XI,
preferably a metal selected from silver, iron, ruthenium, rhodium, nickel, palladium, platinum and/or copper.

44. A process for the production of an oxamide, oxamate or oxalate, comprising the step of:
reacting CO with an amine to form an oxamide or an oxamate or with an alcohol to form an oxalate under flow conditions,
optionally wherein this reaction is performed:
in the presence of a supported homogeneous catalyst, preferably wherein the supported homogeneous catalyst comprises a support and a metal centre coordinated to one or more ligands, wherein the metal centre is bound to the support via one or more of the ligands and wherein the ligand which is bound to the support comprises a phosphorous atom which is bound to the metal centre and/or wherein the metal centre is palladium and/or wherein the support comprises silica, alumina, a transition metal oxide, a polymer, carbon or mixtures thereof, or
in the presence of a heterogeneous catalyst, preferably wherein the heterogeneous catalyst comprises a metal supported on a solid support, in particular wherein the metal is a Group VIII to XI metal and/or wherein the support comprises silica, alumina, a transition metal oxide, a polymer, carbon or mixtures thereof, or
in the presence of a homogeneous catalyst, preferably wherein the homogeneous catalyst comprises a metal centre coordinated to one or more ligands.

45. A process for the production of ethylene glycol comprising the steps of:
reacting CO with an amine to form an oxamide or an oxamate or with an alcohol to form an oxalate under flow conditions; and
reducing the oxamide, oxamate or oxalate to form ethylene glycol, optionally wherein the first step is performed:
in the presence of a supported homogeneous catalyst, preferably wherein the supported homogeneous catalyst comprises a support and a metal centre coordinated to one or more ligands, wherein the metal centre is bound to the support via one or more of the ligands and wherein the ligand which is bound to the support comprises a phosphorous atom which is bound to the metal centre and/or wherein the metal centre is palladium and/or wherein the support comprises silica, alumina, a transition metal oxide, a polymer, carbon or mixtures thereof, or
in the presence of a heterogeneous catalyst, preferably wherein the heterogeneous catalyst comprises a metal supported on a solid support, in particular wherein the metal is a Group VIII to XI metal and/or wherein the support comprises silica, alumina, a transition metal oxide, a polymer, carbon or mixtures thereof, or
in the presence of a homogeneous catalyst, preferably wherein the homogeneous catalyst comprises a metal centre coordinated to one or more ligands,
and/or wherein the second step is a catalytic hydrogenation or wherein the catalyst used in the catalytic hydrogenation comprises one or more metals from Groups VIII to XI,
preferably a metal selected from silver, iron, ruthenium, rhodium, nickel, palladium, platinum and/or copper.

46. A process for the production of polyethylene terephthalate, comprising:
a) producing ethylene glycol according to the process of claim 1 or the following steps of:
reacting CO with an amine to form an oxamide or an oxamate or with an alcohol to form an oxalate under flow conditions; and
reducing the oxamide, oxamate or oxalate to form ethylene glycol, optionally wherein the first step is performed:
in the presence of a supported homogeneous catalyst, preferably wherein the supported homogeneous catalyst comprises a support and a metal centre coordinated to one or more ligands, wherein the metal centre is bound to the support via one or more of the ligands and wherein the ligand which is bound to the support comprises a phosphorous atom which is bound to the metal centre and/or wherein the metal centre is palladium and/or wherein the support comprises silica, alumina, a transition metal oxide, a polymer, carbon or mixtures thereof, or
in the presence of a heterogeneous catalyst, preferably wherein the heterogeneous catalyst comprises a metal supported on a solid support, in particular wherein the metal is a Group VIII to XI metal and/or wherein the support comprises silica, alumina, a transition metal oxide, a polymer, carbon or mixtures thereof, or
in the presence of a homogeneous catalyst, preferably wherein the homogeneous catalyst comprises a metal centre coordinated to one or more ligands, and
b) polymerising the ethylene glycol produced in step a) with terephthalic acid or a terephthalate di-ester to produce polyethylene terephthalate.
